(19) United States
(12) Patent Application Publication
(10) Pub. No.: US 2023/0397861 A1
(43) Pub. Date: Dec. 14, 2023

(54) PIVC-INTEGRATED HEMOLYSIS-REDUCTION ACCESSORIES FOR DIRECT BLOOD DRAW

(71) Applicant: CareFusion 303, Inc., San Diego, CA (US)

(72) Inventors: George MANSOUR, Diamond Bar, CA (US); Bin WANG, Sandy, UT (US); Jon BURKHOLZ, Salt Lake City, UT (US); Curtis BLANCHARD, Herriman, UT (US); Christopher Jessee ZOLLINGER, Chino, CA (US); Megan S. SCHERICH, Salt Lake City, UT (US); Yiping MA, Layton, UT (US)

(21) Appl. No.: 18/330,018

(22) Filed: Jun. 6, 2023

Related U.S. Application Data
(60) Provisional application No. 63/351,565, filed on Jun. 13, 2022.

(51) Int. Cl. A61B 5/15

(52) U.S. Cl. CPC A61B 5/150992; A61B 5/15003; A61B 5/150221

(57) ABSTRACT

Fluid flow devices including a fluid passage for regulating movement of a fluid therethrough, including flow restriction devices having first and second passages that can regulate a fluid flow in a first direction through the device and can regulate a fluid flow in a second direction through the device, and fluid flow restriction devices that can include a first connector having an internal surface defining an inner lumen, a second connector coupled to an end of the first connector, a cannula mounted in the inner lumen extending into the second connector, a lumen of the cannula may define a first flowpath along which a fluid flows into a fluid collection device, and an annulus may be defined between an outer surface of the cannula and the internal surface of the first connector, the annulus may define a second flowpath along which a fluid flows into a catheter assembly.

### CROSS-REFERENCE TO RELATED APPLICATIONS

This application claims the benefit of U.S. Provisional Application No. 63/351,565, titled “PIVC-INTEGRATED HEMOLYSIS-REDUCTION ACCESSORIES FOR DIRECT BLOOD DRAW,” filed Jun. 13, 2022, the disclosure of which is incorporated herein by reference in its entirety.

### TECHNICAL FIELD

The present disclosure generally relates to blood draw and administration of parenteral fluids to a patient, and particularly to systems and methods to reduce hemolysis in PIVC blood draw.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (PIVC). As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe. Alternatively, the blood collection container may include a test tube with a rubber stopper at one end. In some instances, the test tube has had all or a portion of air removed from the test tube so pressure within the test tube is lower than ambient pressure. Such a blood collection container is often referred to as an internal vacuum or a vacuum tube. The blood collection container may also be a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container.

The description provided in the background section should not be assumed to be prior art merely because it is mentioned in or associated with the background section. The background section may include information that describes one or more aspects of the subject technology.

### SUMMARY

The present disclosure provides devices and accessories for reducing hemolysis that can include features for restricting and regulating a fluid flow therethrough. In some instances, the present disclosure provides flow restriction devices that can be attached to a peripheral intravenous catheter.

In some instances, the present disclosure provides flow restriction devices that can regulate a fluid flow moving through the device in one or more direction, such as a first fluid flow moving in a direction away from a patient and a second fluid flow moving in a direction toward the patient.

The present disclosure also provides, in some embodiments, flow restriction devices that are configured to direct a fluid being withdrawn from a patient to move through a first passage, and to direct a fluid being infused toward the patient to move through any of a first and second passage.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1A illustrates an exploded view of a vascular access device including a peripheral intravenous catheter (PIVC) assembly having a flow restriction device and a fluid collection device, in accordance with some embodiments of the present disclosure.

FIG. 1B is an operational illustration of a vascular access device including a peripheral intravenous catheter (PIVC) assembly having a flow restriction device and a fluid collection device, in accordance with some embodiments of the present disclosure.

FIG. 1C is an operational illustration of a vascular access device including a peripheral intravenous catheter (PIVC) assembly having a flow restriction device and a needleless connector, in accordance with some embodiments of the present disclosure.

FIG. 2A illustrates a perspective view of a flow restriction device, in accordance with some embodiments of the present disclosure.

FIG. 2B illustrates a cross-sectional view of the flow restriction device of FIG. 2A, in accordance with some embodiments of the present disclosure

FIG. 2C illustrates an enlarged cross-sectional view of flow channels of the flow restriction device of FIG. 2A, in accordance with some embodiments of the present disclosure.

FIG. 3A illustrates a perspective view of a flow restriction device, in accordance with some embodiments of the present disclosure.

FIG. 3B illustrates a cross-sectional view of the flow restriction device of FIG. 3A, in accordance with some embodiments of the present disclosure

FIG. 3C illustrates an enlarged cross-sectional view of flow channels of the flow restriction device of FIG. 3A, in accordance with some embodiments of the present disclosure.

FIG. 4A illustrates a perspective view of a flow restriction device, in accordance with some embodiments of the present disclosure.

FIG. 4B illustrates a cross-sectional view of the flow restriction device of FIG. 3A, in accordance with some embodiments of the present disclosure.

FIG. 4C illustrates an enlarged partial cross-sectional view of the flow restriction device of FIG. 4A, in accordance with some embodiments of the present disclosure.

FIG. 5A illustrates a perspective view of a flow restriction device, in accordance with some embodiments of the present disclosure.

FIG. 5B illustrates the perspective view of the flow restriction device of FIG. 5A during infusion, in accordance with some embodiments of the present disclosure.

FIG. 5C illustrates the perspective view of the flow restriction device of FIG. 5A during blood draw, in accordance with some embodiments of the present disclosure.

FIG. 6A illustrates a perspective view of a flow restriction device, in accordance with some embodiments of the present disclosure.

FIG. 6B illustrates a cross-sectional view of the flow restriction device of FIG. 6A, in accordance with some embodiments of the present disclosure.

FIG. 6C illustrates an enlarged perspective view of a proximal connector of the flow restriction device of FIG. 6A, in accordance with some embodiments of the present disclosure.

FIG. 7A illustrates a perspective view of a flow restriction device, in accordance with some embodiments of the present disclosure.

FIG. 7B illustrates a cross-sectional view of the flow restriction device of FIG. 7A, in accordance with some embodiments of the present disclosure.

FIG. 8A illustrates a perspective view of a flow restriction device, in accordance with some embodiments of the present disclosure.

FIG. 8B illustrates a cross-sectional view of first and second connectors of the flow restriction device of FIG. 8A, in accordance with some embodiments of the present disclosure.

FIG. 8C illustrates a cross-sectional view of the flow restriction device of FIG. 8A, in accordance with some embodiments of the present disclosure.

FIG. 9A illustrates an exploded view of a vascular access device including a peripheral intravenous catheter (PIVC) assembly having a flow restriction device and a fluid collection device, in accordance with some embodiments of the present disclosure.

FIG. 9B is an operational illustration of a vascular access device including a peripheral intravenous catheter (PIVC) assembly having a flow restriction device and a fluid collection device, in accordance with some embodiments of the present disclosure.

FIG. 9C is an operational illustration of a vascular access device including a peripheral intravenous catheter (PIVC) assembly having a flow restriction device and a needleless connector, in accordance with some embodiments of the present disclosure.

FIG. 10A illustrates a perspective view of a flow restriction device, in accordance with some embodiments of the present disclosure.

FIG. 10B illustrates a cross-sectional view of the flow restriction device of FIG. 10A, in accordance with some embodiments of the present disclosure.

FIG. 11A illustrates a perspective view of a flow restriction device, in accordance with some embodiments of the present disclosure.

FIG. 11B illustrates a cross-sectional view of the flow restriction device of FIG. 11A, in accordance with some embodiments of the present disclosure.

FIG. 12A illustrates a perspective view of a flow restriction device, in accordance with some embodiments of the present disclosure.

FIG. 12B illustrates a perspective view of an insert of the flow restriction device of FIG. 12A, in accordance with some embodiments of the present disclosure.

FIG. 12C illustrates a cross-sectional view of the flow restriction device of FIG. 12A, in accordance with some embodiments of the present disclosure.

FIG. 13A illustrates a perspective view of a flow restriction device, in accordance with some embodiments of the present disclosure.

FIG. 13B illustrates a perspective view of proximal connector of the flow restriction device of FIG. 13A, in accordance with some embodiments of the present disclosure.

FIG. 13C illustrates a cross-sectional view of the flow restriction device of FIG. 13A, in accordance with some embodiments of the present disclosure.

FIG. 14A illustrates a perspective view of a flow restriction device, in accordance with some embodiments of the present disclosure.

FIG. 14B illustrates an exploded view of the flow restriction device of FIG. 14A, in accordance with some embodiments of the present disclosure.

FIG. 14C illustrates a cross-sectional view of a flow restriction device, in accordance with some embodiments of the present disclosure.

FIG. 14D illustrates a cross-sectional view of a flow restriction device when coupled to a fluid collection device, in accordance with some embodiments of the present disclosure.

FIG. 15A illustrates a cross-sectional view of a flow restriction device in a fluid draw position, in accordance with some embodiments of the present disclosure.

FIG. 15B illustrates a cross-sectional view of the flow restriction device of FIG. 15A in a fluid infusion position, in accordance with some embodiments of the present disclosure.

FIG. 15C illustrates a perspective view of a flow-restricting post of the flow restriction device of FIG. 15A, in accordance with some embodiments of the present disclosure.

FIG. 15D illustrates a cross-sectional view of a sliding assembly of the flow restriction device of FIG. 15A, in accordance with some embodiments of the present disclosure.

FIG. 15E illustrates a cross-sectional view of a sliding assembly of the flow restriction device of FIG. 15A, in accordance with some embodiments of the present disclosure.

FIG. 16A illustrates a blood collection system, in accordance with some embodiments of the present disclosure.

FIG. 16B illustrates a blood collection system, in accordance with some embodiments of the present disclosure.

FIG. 17A illustrates a blood collection system, in accordance with some embodiments of the present disclosure.

FIG. 17B illustrates a cross-sectional view of the blood collection system of FIG. 17A, in accordance with some embodiments of the present disclosure.

FIG. 18A illustrates a blood collection system, in accordance with some embodiments of the present disclosure.

FIG. 18B illustrates a cross-sectional view of the blood collection system of FIG. 18A, in accordance with some embodiments of the present disclosure.

FIG. 19A illustrates a perspective view of a blood collection system, in accordance with some embodiments of the present disclosure.

FIG. 19B illustrates a cross-sectional view of the blood collection system of FIG. 19A, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Blood draw via a vascular access device has drawn increasing attention attributed to minimized needle sticks and improved operation efficiency as compared with traditional blood draw methods with venipuncture. Current blood draw using a peripheral intravenous catheter (PIVC) has seen some challenges, one of the most critical is hemolysis related blood quality. In particular, with currently existing PIVC products in the market, along with the standard connection (such as a short extension set and a needleless connector), and blood collection devices (such as a Vacutainer), the shear stress exerted onto blood cells tends to be on the verge of hemolyzing.

Various embodiments of the present disclosure are directed to providing systems and methods to address hemolysis in PIVC blood draw with a hemolysis reduction accessory (also referred to herein as a flow restriction device) which is pre-attached to the PIVC and serves as a flow restrictor to reduce risk of hemolysis. The hemolysis-reduction accessory is advantageously compatible with PIVC placement and does not necessitate change to any of the existing operations. The hemolysis-reduction accessory of the various embodiments described herein is potentially applicable to a wide variety of PIVC products, and compatible with existing blood collection devices and infusion disposables.

Various embodiments of the present disclosure focus on effective flow restriction with the add-on hemolysis-reduction accessory (also referred to herein as a flow restriction device) that regulates the overall flow rate of the entire fluid path as blood cells travel through. The flow restriction device can be either assembled with the PIVC or co-packaged with the PIVC. As such, there is no additional operation during catheter placement since the device has a vented lumen that enables blood flashback. The clinician may connect a blood collection device to the port of the accessory and can then draw blood to the intended volume. After blood draw, the clinician may disconnect and discard the flow restriction device and the blood collection device together. As such, this flow restriction device can be either for single blood draw or stay inline throughout indwell.

Features of the present application can provide flow restriction devices configured for flow of a fluid in two directions, where a flow rate can be different in each direction. In some aspects of the present disclosure, the device can be configured to provide a first flow rate in a first direction, and a second flow rate in a second direction, where the fluid flow is less restricted in the second direction relative to the first direction such that the second flow rate is greater than the first flow rate.

In some embodiments of the present disclosure, a fluid pathway for fluid flow in the first direction is isolated or separated from a fluid pathway for fluid flow in the second direction. In some examples, a fluid flow moving in the first direction moves through a first fluid pathway that is isolated or separated from a second fluid pathway configured for fluid flow in the second direction, and fluid flow moving in the second direction moves through the second fluid pathway and the first fluid pathway.

In some aspects of the present disclosure, the flow restriction device is configured for a fluid to move through a first fluid pathway in the first direction during withdrawal of fluid or blood from a patient to reduce hemolysis of the blood, and for a fluid to move through the first and second fluid pathways in the second direction during infusion of a fluid toward the patient.

In some embodiments, the flow restriction device may integrate a check valve, a flow diverter, an insert, a removable adapter, or another structure that allows hemolysis reduction for fluid moving in the first direction and unobstructed infusion for a fluid moving in the second direction. In some embodiments of the present disclosure, the check valve, flow diverter, insert, removable adapter, or other structure is positioned in a central fluid path of the device.

According to various embodiments of the present disclosure, the flow restriction device may be an insert with luer access and spiral continuous channel on the exterior featuring the flow resistance per design. The flow restriction device may be a plastic insert, as molded, featuring the fluid channel of flow resistance per design. The proximal end of the insert may have a female luer so that a vent plug may be inserted into the flow restriction device that is connected to a port of a luer adapter of the PIVC as packaged to enable blood flashback when the PIVC is placed. In some embodiments, a clinician or other user may remove the vent plug from the flow restriction device and attach a blood collection device to complete the blood draw. In some embodiments, when the clinician connects a blood collection device to this insert, the vent plug may be pushed into a pocket and thereby open the fluid path for blood draw.

Accordingly, the flow restriction devices and systems of the various embodiments described herein are advantageous in that the spiral continuous channel with small (minimized) diameter may increase a length of the fluid pathway defined by the continuous channel or groove through which the blood flows, and may provide increased flow resistance and decreased blood flow rate within the flow resistance device in contrast to the linear internal fluid pathways. As such, a risk of hemolysis during blood collection may advantageously be reduced.

The flow restriction devices and associated blood collection systems of the various embodiments described herein additionally provide further advantages over currently existing blood collection systems. For example, add-on flow restriction devices described herein allow for hemolysis-reduction function to be integrated for PIVC blood draw. Further, the flow restriction devices described herein are compatible with PIVC placement and allow for seamless blood draw at insertion. Furthermore, the flow restriction devices have the potential to stay inline throughout PIVC indwell for multiple blood draws. Additionally, since the flow restriction devices are an add-on which can be easily incorporated without any changes to existing PIVC, there is minimal impact to clinical setting and operations.

An optimized fluid pathway, also referred to herein as a first fluid pathway or flowpath or micro-channel, can be configured for providing a restricted flow rate for reducing hemolysis, and can have features including, but not limited to a tubular fluid pathway, a cannula, a lumen, a continuous non-linear channel, a groove, a fluid channel, and the like.

The fluid pathway can have a length that is selected based on one or more of the following: a gauge of a particular catheter, a particular catheter assembly configuration, or a clinical setup. In some embodiments, the optimized fluid pathway may include a length L from the first luer adapter 14 to the second luer adapter 24. In some embodiments, the optimized fluid pathway may include an inner diameter D.

Fluid flow in a tubular fluid pathway therethrough can be analyzed using Poiseuille's equation:

$$Q = \frac{π {D}^{4} Δ P}{128 μ L} = \frac{Δ P}{{R}_{f}}$$

where ΔP is a change in pressure gradient across the length of the fluid pathway, D and L are the inner diameter and length, respectively, of the fluid pathway, μ is the viscosity of a fluid, and

$${R}_{f} = \frac{128 μ L}{π {D}^{4}}$$

is the fluid resistance. Since y is the viscosity of the fluid and not part of the extension tube geometry, a geometric factor G_{f} is defined such that R_{f} (the fluid resistance) is

$${R}_{f} = \frac{128 μ}{π} {G}_{f} ,$$

where

$${G}_{f} = \frac{L}{{D}^{4}} .$$

In some embodiments, the optimized fluid pathway may have multiple sections with lengths (L1, L2, L3) and inner diameters of (D1, D2, D3), the geometric factor is then:

$${G}_{f} = {\int }_{0}^{L} \frac{dl}{{D ( l )}^{4}}$$

In some embodiments, the optimized fluid pathway may have a cross section that is not circular or complicated inside diameter profile. The geometric factor can be determined by measuring the flow rate (Q) at given pressure (ΔP) with known viscosity (μ) fluid:

$${G}_{f} = \frac{π Δ P}{128 μ Q}$$

The G_{f} value of the optimized fluid pathway may be selected to reduce the max shear stress for each catheter gauge to be the same or less than the max shear stress of a BD 21G VACUTAINER®UltraTouch™ push button blood collection set, which was previously considered the gold standard for blood draws. In some embodiments, G_{f} value of the optimized fluid pathway may be selected to reduce the max shear stress for each catheter gauge to be the same or less than the max shear stress of a BD 25G VACUTAINER®UltraTouch™ push button blood collection set.

In some embodiments, the internal fluid path of the needle assembly 19, extension tube 32, and catheter assembly 37 which may include another extension tube 46 may form the complete fluid path for blood collection. The system geometric factor for the fluid path G_{fs} can be determined in similar fashion as described earlier. In some embodiments, the system geometric factor G_{fs} may be equal to or more than 7.34E+06 (l/in³). In some embodiments, G_{fs} may include another value. In some embodiments, the system geometric factor G_{fs} may be 7.34E+06 (l/in³) plus or minus 10 percent, plus or minus 25 percent, plus or minus 50 percent, or plus or minus 75 percent. In some embodiments, G_{fs} may include another value, which may be selected based on a gauge and/or length of the catheter.

In some embodiments, and by way of non-limiting example, an optimized fluid pathway can have a diameter of approximately 0.014 inch. In another non-limiting example, a cross-sectional area of an optimized fluid pathway is approximately 0.000152 inch².

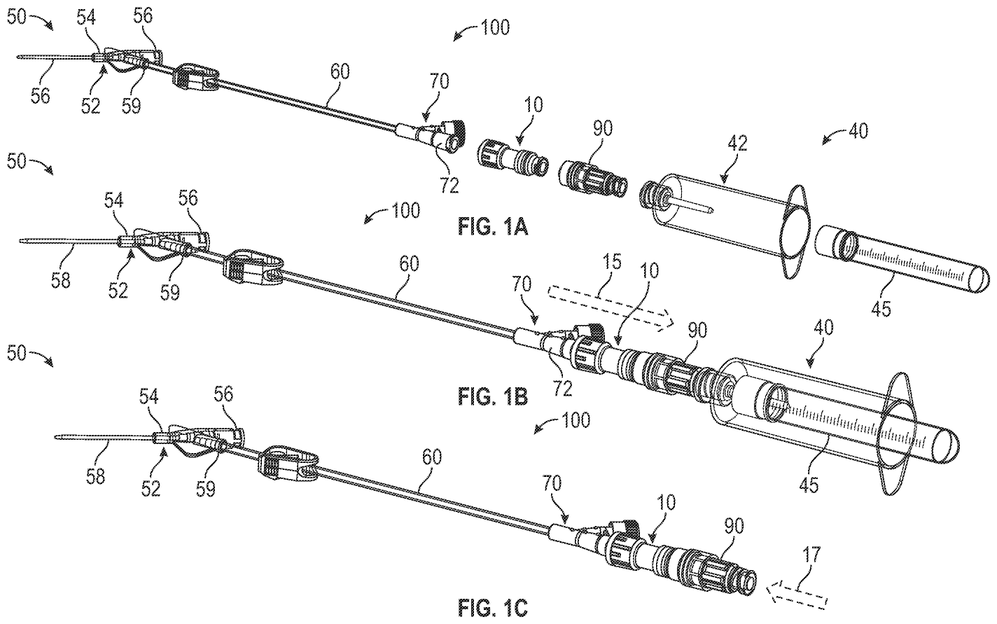

FIGS. 1A-1C illustrate a vascular access device 100 including a peripheral intravenous catheter (PIVC) assembly 50 that includes a flow restriction device 10, in accordance with some embodiments of the present disclosure. FIG. 1A illustrates an exploded view of a vascular access device including a peripheral intravenous catheter (PIVC) assembly having a flow restriction device and a fluid collection device, in accordance with some embodiments of the present disclosure. FIG. 1B is an operational illustration of a vascular access device including a peripheral intravenous catheter (PIVC) assembly having a flow restriction device and a fluid collection device, in accordance with some embodiments of the present disclosure. FIG. 1C is an operational illustration of a vascular access device including a peripheral intravenous catheter (PIVC) assembly having a flow restriction device and a needleless connector, in accordance with some embodiments of the present disclosure.

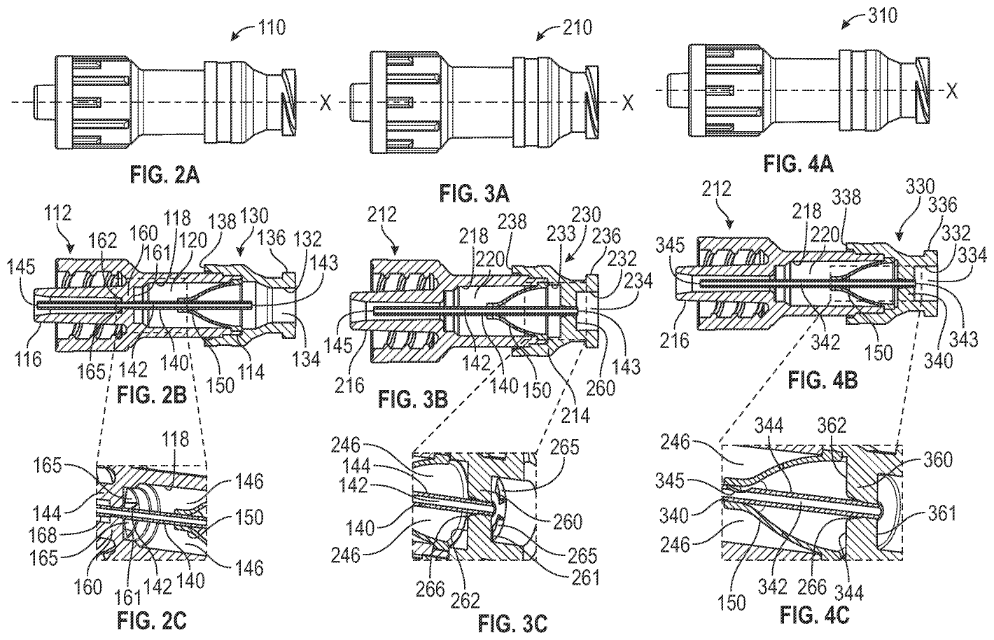

Referring now to FIGS. 1A-1C, flow restriction device 10 is illustrated, according to some embodiments. The flow restriction device 10 may be configured to reduce a likelihood of hemolysis during blood collection using a vascular access device 100. In some embodiments, the vascular access device 100 may include a catheter assembly (e.g., a PIVC) 50. In some embodiments, (as further illustrated in FIGS. 2A-3B), the flow restriction device 10 may include a distal end 12, which may include a body or distal connector 14 configured to couple to the catheter assembly 50. The distal connector 14 may include a male luer connector, or another suitable connector.

In some embodiments, the catheter assembly 50 may include a catheter hub 52, which may include a distal end 54, a proximal end 56, and a lumen extending through the distal end and the proximal end. The catheter assembly 50 may further include a catheter 58, which may be secured within the catheter hub 52 and may extend distally from the distal end 54 of the catheter hub 52. In some embodiments, the catheter may be a peripheral intravenous catheter (PIVC).

In some embodiments, the catheter assembly 50 may include or correspond to any suitable catheter assembly 50. In some embodiments, the catheter assembly 50 may be integrated and include an extension tube 60, which may extend from and be integrated with a side port 59 of the catheter hub 52. A non-limiting example of an integrated catheter assembly is the BD NEXIVA™ Closed IV Catheter system, available from Becton Dickinson and Company. In some embodiments, a proximal end of the extension tube 60 may be coupled to an adapter 70, such as, for example, a Y-adapter or single port luer adapter. In some embodiments, the distal connector 14 of flow restriction device 10 may be configured to couple to the Y-adapter 70.

In some embodiments, the catheter assembly 50 may be non-integrated and may not include the extension tube 60. In these and other embodiments, the flow restriction device 10 may be configured to couple to the proximal end 56 of the catheter hub 52 or another suitable portion of the catheter assembly 50. In some embodiments, the catheter assembly 50 may be coupled to a removable extension tube 60. In some embodiments, the flow restriction device 10 may be coupled directly to the catheter adapter, eliminating the extension tube and providing a compact catheter system.

FIG. 2A illustrates a perspective view of a flow restriction device 110, in accordance with some embodiments of the present disclosure. FIG. 2B illustrates a cross-sectional view of the flow restriction device 110 of FIG. 2A, in accordance with some embodiments of the present disclosure FIG. 2C illustrates an enlarged cross-sectional view of flow channels 165 of the flow restriction device of FIG. 2A, in accordance with some embodiments of the present disclosure.

As illustrated in FIG. 2A, with continued reference to FIGS. 1A and 1B, in some embodiments, the flow restriction device 110 may include a first connector 112 configured to couple to the catheter assembly 50. The first connector 112 may have a proximal end 114, a distal end 116, and an internal surface 118 defining an inner lumen 120 of the first connector 112. As depicted, the first connector 112 may further include a support portion 160 disposed between the proximal and distal ends 114 and 116. The support portion 160 may have a proximal end 161, a distal end 162, and may include a central mounting aperture 165 extending from the proximal end 161 to the distal end 162. In some embodiments, the support portion 160 may further include a plurality of fluid channels 165 disposed radially outward of and surrounding the central mounting aperture 165. The plurality of fluid channels 165 may extend from the proximal end 161 to the distal end 162 of the support portion 160 to fluidly communicate the inner lumen 120 with the catheter assembly.

In some embodiments, the flow restriction device 110 may further include a second connector 130 coupled to the proximal end 114 of the first connector 112. The second connector 130 may be configured to couple to fluid collection device 40 (e.g., a blood collection device). For example, the second connector 130 may be integrated with the blood collection device 40 or monolithically formed with the blood collection device 40 as a single unit. As another example, the second connector 130 may be in the form of a female luer connector or another suitable connector, which may be coupled with a male luer portion of the blood collection device 40. The second connector 130 may include a lumen 134 extending therethrough for coupling to the male luer portion of the blood collection device 40.

In accordance with various embodiments of the present disclosure, the flow restriction device 110 may further include a cannula 140 mounted in the inner lumen 120 of the first connector 112. As depicted, the cannula 140 may extend from the distal end 116 of the first connector 112 into the second connector 130. The cannula may have a proximal end 143, a distal end 145, and a lumen 142 extending therethrough. The lumen 142 may define a first flowpath or micro-channel along which a fluid may flow from the distal end 116 toward the proximal end 114, such as from the distal end 116 to the proximal end 114 and into the fluid collection device 40 coupled thereto. A fluid may also flow through first flowpath in a direction from the proximal end 114 toward the distal end 116. In some aspects, a fluid may also flow through first flowpath and through a second flowpath when moving in a direction from the proximal end 114 toward the distal end 116.

As depicted, the cannula 140 may be mounted in the central mounting aperture 166 of the first connector support portion 160 and may fluidly communicate the cannula 140 with the catheter assembly 50. For example, in some embodiments, a leg 72 of the Y-adapter 70 may be coupled to the flow restriction device 110. For example, the leg 72 of Y-adapter 70 may include a lumen into which the distal end 116 of the first connector 112 having the cannula 140 mounted therein may be coupled. The Y-adapter 70 may fluidly communicate the flow restriction device 110, and cannula 140 mounted therein, with the catheter assembly 50, for example, via the extension tubing 60. Accordingly, the lumen 142 of the cannula 140 may define a linear fluid pathway with a reduced, small, or micro-sized diameter (as discussed below) through which fluid entering the flow restriction device 110 from the catheter assembly, may flow through the flow restriction device 110 for collection in the fluid collection device 40. For example, where blood is being withdrawn or collected from a patient, the medical fluid 15 may be blood, and the fluid collection device 40 may be a blood collection device. In some embodiments, the blood collection device may be a luer lock access device (LLAD). Accordingly, during blood collection or withdrawal from the patients, the blood sample 15 may flow from the distal end 116 of the first connector 112 into the LLAD 40 via the first flowpath or micro-channel.

In some embodiments, the cannula 140 may be an elongate, thin tube with the lumen having a small, reduced or micro-sized diameter. For example, in some embodiments, the lumen 142 of the cannula 140, that defines the first flowpath or micro-channel along which fluid may flow from the distal end 116 into the fluid collection device 40 via the second connector 130. The cannula 140 can define a lumen 142 formed by any of a length, a diameter, and a cross-sectional area as described above with reference to an optimized fluid pathway.

When the fluid is blood being withdrawn from a patient, blood cells may experience shear stress as they flow from the catheter assembly 50 into the blood collection device 40. For example, the maximum shear stress may be along the wall of the blood cell, often referred to as wall shear stress. Wall shear stress on blood cells is considered a major source of mechanical damage to blood cells causing hemolysis of the blood cells. In some embodiments, the lumen 142 of the cannula 140 having a reduced or micro-sized diameter may facilitate increased flow resistance within the vascular access system 100 to distribute the pressure differential and reduce shear stress experienced by the red blood cells of the blood 15. The minimized diameter of the first fluid pathway or micro-channel defined by the lumen 142 of the cannula 140 may provide increased resistance to flow of the blood 15 and thereby decrease blood flow rate within the flow resistance device 110. Since the decreased blood flow rate causes a reduction in shear stress experienced by the red blood cells of the blood 15, a risk of hemolysis during blood collection may advantageously be reduced.

In some embodiments, as depicted in FIG. 2C, an annulus 146 may be defined between an outer surface of the cannula 140 and the internal surface 118 of the first connector 112. The annulus 146 may define a second flowpath along which a fluid flows from the lumen 134 of the second connector 130 and into the catheter assembly 50 via the first connector 116. The plurality of fluid channels 165 may extend from the proximal end 161 to the distal end 162 of the support portion 160 to fluidly communicate the annulus 146 with the catheter assembly. Accordingly, the plurality of fluid channels 165 may define at least a portion of the second flowpath.

According to various embodiments of the present disclosure, the flow restriction device 110 may further include a check valve 150 mounted in the annulus 146 at the proximal end 114 of the first connector 112. As depicted, the check valve 150 may be sleeved over at least a portion of the cannula 140. The check valve 150 may have a shape or structure configured to prevent fluid flowing from the distal end 116 of the first connector 112 into the fluid collection device 40 via the second flowpath, while allowing fluid to flow from the second connector 130 into the first connector 112 and the catheter assembly 50 via the second flowpath. In some embodiments, the fluid flowing from the second connector 130 into the first connector 112 and the catheter assembly via the second flowpath may be an IV fluid 17. Accordingly, the blood 15 containing blood cells may be forced to flow through the micro-channel fluid pathway defined by the lumen 142 of the cannula 140 in order to arrive at the blood collection device 40, while the IV fluid 36 may flow to the catheter assembly 50 through the second flowpath including the annulus 146 and the plurality of fluid channels 165.

In some embodiments, a cross-sectional flow area of the annulus 146 may be larger than a cross-sectional flow area of the lumen 142. For example, a cross-sectional area of the annulus 146 about a plane transverse to a central longitudinal axis X of the first connector 112 may be greater than a cross-sectional area of the lumen 142 about the plane. The cannula 140 can define a lumen 142 formed by any of a length, a diameter, and a cross-sectional area as described above with reference to an optimized fluid pathway.

By way of non-limiting example, in some embodiments, the lumen 142 can comprise a diameter of approximately 0.014 inch. In some examples, a cross-sectional area of the lumen 142 is approximately 0.000152 inch² and the cross-sectional area of the annulus 146 is approximately 0.0363 inch². In some embodiments, a diameter of the lumen 142 of the cannula 140 may be smaller than a thickness of a lumen of the annulus 146. However, the various embodiments of the present disclosure are not limited to the aforementioned configurations.

The aforementioned configuration is advantageous in that the reduced or minimized diameter or size of the micro-channel fluid pathway defined by lumen 142 of the cannula 140 may provide the increased flow resistance and decreased blood flow rate within the flow resistance device 110 in contrast to if the blood were to flow into the blood collection device 40 via the plurality of fluid channels 165 and the annulus 146 of the second flowpath. Accordingly, a risk of hemolysis during blood collection may advantageously be reduced. However, the larger size of the cross-sectional flow area of the annulus 146 as compared with the cross-sectional flow area of the lumen 142 may provide an additional advantage of allowing an unrestricted and increased amount of the IV fluid 17 to flow to the patient via the second flowpath versus via the reduced or minimized diameter or size of the micro-channel fluid pathway defined by lumen 142 of the cannula 140. Accordingly, the IV fluid 36 may flow in a second unrestricted (less flow resistance) direction (proximal to distal), toward the catheter assembly 50, opposite from the first direction (distal to proximal) in which the blood sample 15 having blood cells flows.

FIG. 3A illustrates a perspective view of a flow restriction device 210, in accordance with some embodiments of the present disclosure. FIG. 3B illustrates a cross-sectional view of the flow restriction device 210 of FIG. 3A, in accordance with some embodiments of the present disclosure. FIG. 3C illustrates an enlarged cross-sectional view of flow channels 245 of the flow restriction device 210 of FIG. 3A, in accordance with some embodiments of the present disclosure.

As illustrated in FIG. 3A, with continued reference to FIGS. 1A and 1B, in some embodiments, the flow restriction device 210 may include a first connector 212 configured to couple to the catheter assembly 50. The first connector 212 may have a proximal end 214, a distal end 216, and an internal surface 218 defining an inner lumen 220 of the first connector 212. In some embodiments, the flow restriction device 210 may further include a second connector 230 coupled to the proximal end 214 of the first connector 212. The second connector 230 may be configured to couple to a fluid collection device 40 (e.g., a blood collection device). For example, the second connector 230 may be integrated with the blood collection device 40 or monolithically formed with the blood collection device 40 as a single unit. As another example, the second connector 230 may be in the form of a female luer connector or another suitable connector, which may be coupled with a male luer portion of the blood collection device 40. The second connector 230 may include a lumen 234 extending therethrough for coupling to the male luer portion of the blood collection device 40.

In some embodiments, the second connector 230 may further include a proximal end 236, a distal end 238, and a support portion 260 disposed between the proximal and distal ends 236 and 238. The support portion 260 may have a proximal end 261, a distal end 262, and may include a central mounting aperture 266 extending from the proximal end 261 to the distal end 262. In some embodiments, the support portion 260 may further include a plurality of fluid channels 265 disposed radially outward of and surrounding the central mounting aperture 266. The plurality of fluid channels 265 may extend from the proximal end 261 to the distal end 262 of the support portion 260 to fluidly communicate the lumen 234 of the second connector 230 with the inner lumen 220 of the first connector 212, which is fluidly coupled to the catheter assembly 50. Accordingly, the plurality of fluid channels 265 may define at least a portion of the second flowpath.

In accordance with various embodiments of the present disclosure, the flow restriction device 210 may further include a cannula 140 mounted in the lumen 234 of the second connector 230. In particular, the cannula 140 may have a proximal end 143, a distal end 145, and a lumen 142 extending therethrough. As depicted, the proximal end 143 of the cannula 140 may be mounted in the central mounting aperture 266 for fluidly communicating the cannula 140 with the fluid collection device 40. The cannula 140 may extend from the lumen 234 of the second connector 230 into the first connector 212. The cannula 140 may have a proximal end 143, a distal end 145, and a lumen 142 extending therethrough. The lumen 142 may define a first flowpath or micro-channel along which a fluid may flow between the proximal end 214 and the distal end 216 of the first connector 212. As depicted, the cannula 140 may be mounted in the central mounting aperture 266 of the second connector support portion 260 and may fluidly communicate the cannula 140 with the catheter assembly 50. For example, in some embodiments, a leg 72 of the Y-adapter 70 may be coupled to the flow restriction device 210. For example, the leg 72 of Y-adapter 70 may include a lumen into which the distal end 216 of the first connector 212 having the cannula 140 disposed therein may be coupled. The Y-adapter 70 may fluidly communicate the flow restriction device 210, and cannula 140 mounted therein, with the catheter assembly 50, for example, via the extension tubing 60. Accordingly, the lumen 142 of the cannula 140 may define a linear fluid pathway with a reduced, small, or micro-sized diameter (as discussed below) through which fluid entering the flow restriction device 210 from the catheter assembly, may flow through the flow restriction device 210 for collection in the fluid collection device 40. For example, where blood is being withdrawn or collected from a patient, the medical fluid 15 may be blood, and the fluid collection device 40 may be a blood collection device. In some embodiments, the blood or fluid collection device may be a luer lock access device (LLAD). Accordingly, during blood collection or withdrawal from the patient, the blood sample 15 may flow from the distal end 216 of the first connector 212 into the LLAD 40 via the first flowpath or micro-channel.

In some embodiments, the cannula 140 may be an elongate, thin tube with the lumen having a small, reduced or micro-sized diameter. For example, in some embodiments, the lumen 142 of the cannula 140, that defines the first flowpath or micro-channel along which fluid may flow from the distal end 116 into the fluid collection device 40 via the second connector 130. The cannula 140 can define a lumen 142 formed by any of a length, a diameter, and a cross-sectional area as described above with reference to an optimized fluid pathway.

When the fluid 15 is blood being withdrawn from a patient, blood cells may experience shear stress as they flow from the catheter assembly 50 into the blood collection device 40. For example, the maximum shear stress may be along the wall of the blood cell, often referred to as wall shear stress. Wall shear stress on blood cells is considered a major source of mechanical damage to blood cells causing hemolysis of the blood cells. In some embodiments, the lumen 142 of the cannula 140 having a reduced or micro-sized diameter may facilitate increased flow resistance within the vascular access system 100 to distribute the pressure differential and reduce shear stress experienced by the red blood cells of the blood 15. The minimized diameter of the first fluid pathway or micro-channel defined by the lumen 142 of the cannula 140 may provide increased resistance to flow of the blood 15 and thereby decrease blood flow rate within the flow resistance device 210. Since the decreased blood flow rate causes a reduction in shear stress experienced by the red blood cells of the blood 15, a risk of hemolysis during blood collection may advantageously be reduced.

In some embodiments, as depicted in FIG. 3C, an annulus 246 may be defined between an outer surface of the cannula 140 and the internal surface 118 of the first connector 212. The annulus 246 may define a second flowpath along which a fluid flows from the lumen 234 of the second connector 230 and into the catheter assembly 50 via the first connector 216. The plurality of fluid channels 265 may extend from the proximal end 261 to the distal end 262 of the support portion 260 to fluidly communicate the annulus 246 with the catheter assembly 50. Accordingly, the plurality of fluid channels 265 may define at least a portion of the second flowpath.

Similar to the flow restriction device 110, the flow restriction device 210 may further include a check valve 150 mounted in the annulus 246 at the proximal end 214 of the first connector 212. As depicted, the check valve 150 may be sleeved over at least a portion of the cannula 140. The check valve 150 may have a shape or structure configured to prevent fluid flowing from the distal end 216 of the first connector 212 into the fluid collection device 40 via the second flowpath, while allowing fluid to flow from the second connector 230 into the first connector 212 and the catheter assembly 50 via the second flowpath. In some embodiments, the fluid flowing from the second connector 230 into the first connector 212 and the catheter assembly 50 via the second flowpath may be an IV fluid 17. Accordingly, the blood 15 containing blood cells may be forced to flow through the micro-channel fluid pathway defined by the lumen 142 of the cannula 140 in order to arrive at the blood collection device 40, while the IV fluid 17 may flow to the catheter assembly 50 through the second flowpath including the annulus 246 and the plurality of fluid channels 265.

In some embodiments, a cross-sectional flow area of the annulus 246 may be larger than a cross-sectional flow area of the lumen 142. For example, a cross-sectional area of the annulus 246 about a plane transverse to a central longitudinal axis X of the first connector 212 may be greater than a cross-sectional area of the lumen 142 about the plane. The cannula 140 can define a lumen 142 formed by any of a length, a diameter, and a cross-sectional area as described above with reference to an optimized fluid pathway. In some embodiments, diameter of the lumen 142 of the cannula 140 may be smaller than a thickness of a lumen of the annulus 246.

The aforementioned configuration is advantageous in that the reduced or minimized diameter or size of the micro-channel fluid pathway defined by lumen 142 of the cannula 140 may provide the increased flow resistance and decreased blood flow rate within the flow resistance device 210 in contrast to if the blood were to flow into the blood collection device 40 via the plurality of fluid channels 265 and the annulus 246 of the second flowpath. Accordingly, a risk of hemolysis during blood collection may advantageously be reduced. However, the larger size of the cross-sectional flow area of the annulus 246 as compared with the cross-sectional flow area of the lumen 142 may provide an additional advantage of allowing an unrestricted and increased amount of the IV fluid 17 to flow to the patient via the second flowpath versus via the reduced or minimized diameter or size of the micro-channel fluid pathway defined by lumen 142 of the cannula 140. Accordingly, the IV fluid 17 may flow in a second unrestricted (less flow resistance) direction (proximal to distal), toward the catheter assembly 50, opposite from the first direction (distal to proximal) in which the blood sample 15 having blood cells flows.

FIG. 4A illustrates a perspective view of a flow restriction device 310, in accordance with some embodiments of the present disclosure. FIG. 4B illustrates a cross-sectional view of the flow restriction device of FIG. 4A, in accordance with some embodiments of the present disclosure. FIG. 4C illustrates an enlarged partial cross-sectional view of the flow restriction device of FIG. 4A, in accordance with some embodiments of the present disclosure. In accordance with some embodiments, the flow restriction device 310 is similar to the flow restriction device 210, and where the elements are the same, the numbering of reference characters has been maintained, and a detailed description of these similar elements is omitted herein.

The flow restriction device 310 may differ from the flow restriction device 210 with respect to the support portion 360 and the cannula 340, as shall be described in further detail below.

Similar to the second connector 230 of the flow restriction device 210, the second connector 330 of the flow restriction device 310 may include a proximal end 336, a distal end 338, and a support portion 360 disposed between the proximal and distal ends 336 and 338. The support portion 360 may have a proximal end 361, a distal end 362, and may include a central mounting aperture 366 extending from the proximal end 261 to the distal end 262. However, in contrast to the support portion 260 of the second connector 230 of flow restriction device 210, the support portion 360 of the second connector 330 of flow restriction device 310 may not include a plurality of fluid channels disposed radially outward of and surrounding the central mounting aperture 366.

In some embodiments, the flow restriction device 310 may include a cannula 340 mounted in the lumen 334 of the second connector 330. In particular, the cannula 340 may have a proximal end 343, a distal end 345, and a lumen 342 extending therethrough. As depicted, the proximal end 343 of the cannula 340 may be mounted in the central mounting aperture 366 for fluidly communicating the cannula 340 with the fluid collection device 40. The cannula 340 may extend from the lumen 334 of the second connector 330 into the first connector 212. The cannula 340 may have a proximal end 343, a distal end 345, and a lumen 342 extending therethrough.

The lumen 342 of cannula 340 may define a first flowpath or micro-channel along which a fluid may flow from the distal end 216 of the first connector 212 into the fluid collection device 40. As depicted, the cannula 340 may be mounted in the central mounting aperture 266 of the second connector support portion 260 and may fluidly communicate the cannula 340 with the catheter assembly 50. For example, in some embodiments, a leg 72 of the Y-adapter 70 may be coupled to the flow restriction device 310. For example, the leg 72 of Y-adapter 70 may include a lumen into which the distal end 216 of the first connector 212 having the cannula 340 disposed therein may be coupled. The Y-adapter 70 may fluidly communicate the flow restriction device 310, and cannula 340 mounted therein, with the catheter assembly 50, for example, via the extension tubing 60. Accordingly, the lumen 342 of the cannula 340 may define a linear fluid pathway with a reduced, small, or micro-sized diameter (as discussed below) through which fluid entering the flow restriction device 310 from the catheter assembly, may flow through the flow restriction device 310 for collection in the fluid collection device 40. For example, where blood is being withdrawn or collected from a patient, the medical fluid 15 may be blood, and the fluid collection device 40 may be a blood collection device. In some embodiments, the blood collection device may be a luer lock access device (LLAD). Accordingly, during blood collection or withdrawal from the patient, the blood sample 15 may flow from the distal end 216 of the first connector 212 into the LLAD 40 via the first flowpath or micro-channel.

In some embodiments, the cannula 340 may be an elongate, thin tube with the lumen having a small, reduced or micro-sized diameter. For example, in some embodiments, the lumen 342 of the cannula 340, that defines the first flowpath or micro-channel along which fluid may flow from the distal end 216 into the fluid collection device 40 via the second connector 330. The cannula 340 can define a lumen 342 formed by any of a length, a diameter, and a cross-sectional area as described above with reference to an optimized fluid pathway.

The cannula 340 may be similar in configuration to the cannula 140, with the difference that in some embodiments, the cannula 340 may include a notch 345 along a length of the cannula 340 between the proximal and distal ends 343 and 345 of the cannula 340. In particular, the notch 345 may be disposed at a position corresponding to a distal end of the check valve 150. Accordingly, in an open configuration of the check valve 150, for example during infusion of a fluid (e.g., IV fluid) from the proximal end 343 of the cannula, the notch 345 may fluidly connecting the lumen 342 of the cannula 340 with the annulus 246. As such, at least a portion of the infusion fluid may flow from the proximal end 343 of the cannula 340 into the annulus 246 via the notch 345 of the cannula 340.

In some embodiments, a cross-sectional flow area of the annulus 246 may be larger than a cross-sectional flow area of the lumen 342. For example, a cross-sectional area of the annulus 246 about a plane transverse to a central longitudinal axis X of the first connector 212 may be greater than a cross-sectional area of the lumen 342 about the plane. The cannula 140 can define a lumen 142 formed by any of a length, a diameter, and a cross-sectional area as described above with reference to an optimized fluid pathway. In some embodiments, a diameter of the lumen 342 of the cannula 340 may be smaller than a thickness of a lumen of the annulus 246.

The aforementioned configuration is advantageous in that the reduced or minimized diameter or size of the micro-channel fluid pathway defined by lumen 342 of the cannula 340 may provide the increased flow resistance and decreased blood flow rate within the flow resistance device 310 in contrast to if the blood were to flow into the blood collection device 40 via the annulus 246 of the second flowpath. Accordingly, a risk of hemolysis during blood collection may advantageously be reduced. However, the larger size of the cross-sectional flow area of the annulus 246 as compared with the cross-sectional flow area of the lumen 342 may provide an additional advantage of allowing an unrestricted and increased amount of the IV fluid 17 to flow to the patient from the lumen and the notch 345 via the second flowpath versus via the reduced or minimized diameter or size of the micro-channel fluid pathway defined by lumen 342 of the cannula 340. Accordingly, the IV fluid 17 may flow in a second unrestricted (less flow resistance) direction (proximal to distal), toward the catheter assembly 50, opposite from the first direction (distal to proximal) in which the blood sample 15 having blood cells flows.

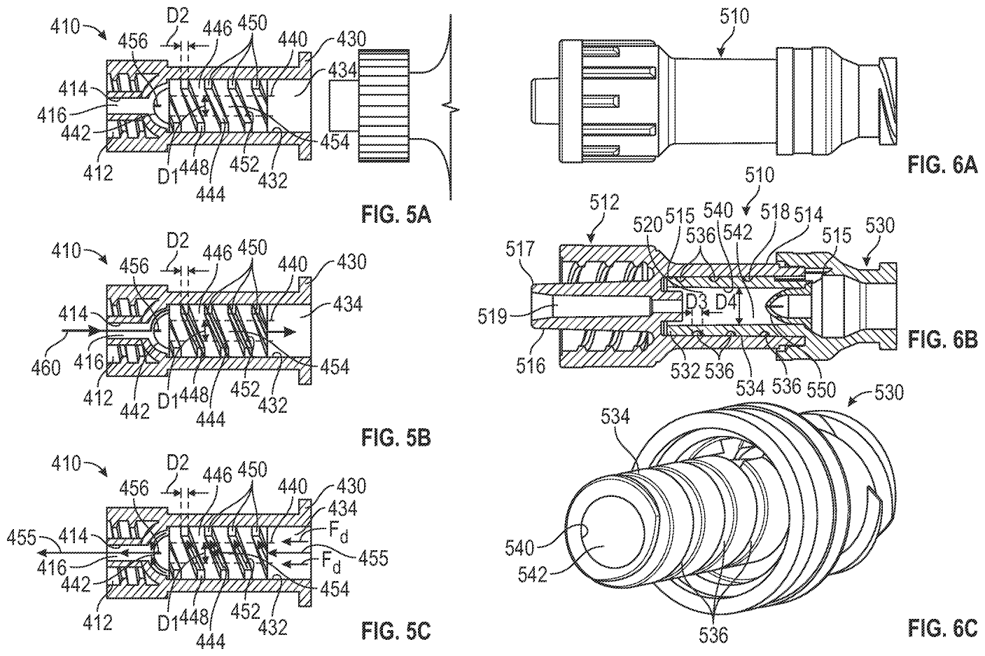

FIG. 5A illustrates a perspective view of a flow restriction device 410, in accordance with some embodiments of the present disclosure. FIG. 5B illustrates the perspective view of the flow restriction device 410 of FIG. 5A during blood draw, in accordance with some embodiments of the present disclosure. FIG. 5C illustrates the perspective view of the flow restriction device 410 of FIG. 5A during infusion, in accordance with some embodiments of the present disclosure. As illustrated in FIGS. 5A-5B, with continued reference to FIGS. 1A and 1B, in some embodiments, the flow restriction device 410 may include a distal connector portion 412 configured to couple to a catheter assembly 50, and a proximal connector portion 430 extending proximally from the distal connector portion 412, and configured to couple to a fluid collection device 40. The distal connector portion 412 may include an internal surface 414 defining a lumen 416 thereof, and the proximal connector portion 430 may include an internal surface 432 defining a lumen 434 fluidly connected to the lumen 416 of the distal connector portion 412. As depicted, the flow restriction device 410 may further include a plug 444 disposed in the lumen 434 of the proximal connector portion 430.

According to various embodiments of the present disclosure, the plug 444 may include a head portion 442 and a body portion 448 extending proximally from the head portion 442. As depicted, the head portion may include a slit 456, and the body portion 448 may include a plurality of threads 446 extending along an outer surface of the body portion 448. The plug 444 may further include an inner surface 452 defining a lumen 454 of the plug 444. In some embodiments, the lumen 454 may define an internal flowpath 455 in which fluid may flow from the proximal connector portion 430 into the catheter assembly 50 via the distal connector portion.

As depicted, the internal surface 432 of the proximal connector portion 430 may encase, surround, or otherwise envelope the outer surface of the body portion 448 to define a continuous non-linear channel 450 along spacing between adjacent threads of the plurality of threads 446. In some embodiments, the continuous non-linear channel 450 may be in the form of a continuous groove having a coil shape recessed in the outer surface of the body portion 448. In some embodiments, the continuous non-linear channel 450 may form a coil shape, an S-shape, or another suitable non-linear, winding shape. For example, the continuous non-linear channel 450 may have a coil shape (which may include a spiral) or an S-shape recessed in the outer surface of the body portion 448. The aforementioned configuration is advantageous in that the spiral continuous non-linear channel 450—by virtue of its wrapping around the outer surface of the body portion 448—may increase a length of the fluid pathway defined by the continuous non-linear channel 450 through which the blood sample 15 flows as compared to a linear channel. Where the medical fluid being withdrawn from a patient is blood, blood cells may experience shear stress as they flow from the catheter assembly 50 into the blood collection device 40. In some embodiments, the continuous non-linear channel 450 may facilitate increased flow resistance within the vascular access system 100 to distribute the pressure differential and reduce shear stress experienced by the red blood cells of the blood 15.

Additionally, in some embodiments, the continuous non-linear channel 450 may have a first diameter D1, and the lumen 454 of the plug 444 may have a second diameter D2, and the first diameter D1 may be smaller than the second diameter D2. The continuous non-linear channel 450 can be formed by any of a length, a diameter, and a cross-sectional area as described above with reference to an optimized fluid pathway.

As a non-limiting example, in some embodiments, first diameter D1 may range from about 0.02 inches to 0.03 inches, in some instances range from about 0.022 inches to 0.028 inches, more typically from about 0.024 inches to 0.026 inches, and in some embodiments approximately 0.025 inches. Though recited in terms of certain ranges, it will be understood that all ranges from the lowest of the lower limits to the highest of the upper limits are included, including all intermediate ranges or specific angles, within this full range or any specifically recited range. However, the various embodiments of the present disclosure are not limited to the aforementioned configuration.

The aforementioned configuration is advantageous in that due to the minimized diameter or size of the fluid pathway defined by continuous non-linear channel 450, the fluid pathway defined by continuous non-linear channel 450 may thereby provide increased flow resistance and decreased blood flow rate within the flow resistance device 410 in contrast to the internal flowpath 455 defined in the lumen 454 of the plug 444. Accordingly, a risk of hemolysis during blood collection may advantageously be reduced.

The aforementioned configuration with the second diameter D2 being larger than the first diameter D1 may further be advantageous in further allowing an unrestricted and increased amount of the IV fluid 17 to flow to the patient via (i) the larger diameter internal flowpath 445 and (ii) the reverse fluid pathway defined by continuous non-linear channel 450 (illustrated in FIG. 5C), versus flowing only in the smaller (minimized) diameter fluid pathway defined by continuous non-linear channel 450. Accordingly, the IV fluid 17 may flow in a second unrestricted (less flow resistance) direction (proximal to distal) opposite from the first direction (distal to proximal) in which the blood sample 15 having blood cells flows.

In operation, during blood collection or withdrawal from the patients, the blood 15 may flow from the patient's vein into the catheter assembly 50, through the extension tubing 60, enter the distal connector portion 412 of the flow restriction device 410 via the lumen 416 and due to the presence of the plug 444 in the lumen 434 of the proximal connector portion 430. The slit 456 of the head portion can be closed or nominally closed such that movement of the blood through the slit 456 is resisted. The blood 15 may be forced by the head portion 442 and the body portion 448 to flow around the outer surface of the plug 444 into the continuous non-linear channel 450, and exit the flow restriction device 410 into the blood collection device 40. Accordingly, during blood collection or withdrawal from the patients, the blood 15 may flow into the blood collection device 40 via the continuous non-linear channel 450 having minimal diameter.

The flow restriction device 410 of the various embodiments described herein is advantageous over currently existing blood collection systems. For example, during blood draw with currently existing blood draw devices, blood cells may experience shear stress as they flow from the distal end to the proximal end of the blood collection systems. The maximum shear stress may be along the wall of the blood cell, often referred to as wall shear stress. Wall shear stress on blood cells is considered a major source of mechanical damage to blood cells causing hemolysis of the blood cells. In some embodiments, the continuous non-linear channel 450 having minimal diameter may facilitate increased flow resistance within the vascular access system 100 to distribute the pressure differential and reduce shear stress experienced by the red blood cells of the blood 15. For example, the minimized diameter of the continuous non-linear channel 450 may provide increased resistance to flow of the blood 15 and thereby decrease blood flow rate within the flow resistance device 410. Since the decreased blood flow rate causes a reduction in shear stress experienced by the red blood cells of the blood 15, a risk of hemolysis during blood collection may advantageously be reduced.

FIG. 6A illustrates a perspective view of a flow restriction device 510, in accordance with some embodiments of the present disclosure. FIG. 6B illustrates a cross-sectional view of the flow restriction device 510 of FIG. 6A, in accordance with some embodiments of the present disclosure. FIG. 6C illustrates an enlarged perspective view of a proximal connector 530 of the flow restriction device of FIG. 6A, in accordance with some embodiments of the present disclosure. As illustrated in FIGS. 6A-6C, with continued reference to FIGS. 1A and 1B, in some embodiments, the flow restriction device 510 may include a distal connector 512 configured to couple to a catheter assembly 50, and a proximal connector 530 coupled to the distal connector 512 and configured to couple to a fluid collection device 40. Accordingly, during draw of a fluid (e.g., blood draw), the fluid may flow from the catheter assembly 50 into the flow restriction device 510 (e.g., via the extension tubing 60), and exit the flow restriction device 510 into the blood collection device 40. In some embodiments, the distal connector 512 may include a first connection portion 514 at a proximal end thereof and a second connection portion 516 at a distal end thereof. As depicted, the first connection portion 514 may be in the form of a cylindrical body having an inner surface 515 defining a lumen 520 of the distal connector 512. The lumen may be configured such that at least a portion of the proximal connector 530 may be inserted, fitted, or otherwise coupled therein to fluidly couple the distal and proximal connectors 512 and 530. For example, in some embodiments the proximal connector 530 may include an insert portion 534 for inserting into the lumen 520 of the first connection portion 514. As depicted, an outer surface of the insert portion 534 may include a continuous non-linear channel 536 recessed therein. In the coupled or assembled configuration of the of the distal connector 512 and the proximal connector 530, the inner surface 515 of the first connection 514 portion may encase, surround, or otherwise envelope the outer surface of the insert portion 534 such that the continuous non-linear channel 536 and the inner surface 515 of the first connection portion 514 define a non-linear fluid pathway along which a fluid (e.g., blood) may flow from the distal connector 512 into the fluid collection device 40 via the proximal connector 530.

In some embodiments, the continuous non-linear channel 536 may form a coil shape, an S-shape, or another suitable non-linear, winding shape. For example, the continuous non-linear channel 536 may have a coil shape (which may include a spiral) recessed in the outer surface of the insert portion 534. In some embodiments, the continuous non-linear channel 536 may have an S-shape recessed in the outer surface of the insert portion 534. The aforementioned configuration is advantageous in that the spiral, coil shape, S-shape, or other suitable non-linear, winding shape of the continuous non-linear channel 536—by virtue of its wrapping around the outer surface of the insert portion 534—may increase a length of the fluid pathway defined by the continuous non-linear channel 536 through which the blood sample flows.

Where the medical fluid is blood being withdrawn or collected from a patient, the medical fluid may be a blood sample, and the fluid collection device 40 may be a blood collection device. In some embodiments, the blood collection device may be a luer lock access device (LLAD). Where the medical fluid is blood being withdrawn from a patient, blood cells may experience shear stress as they flow from the distal connector 512 to the proximal connector 530 of the flow restriction device 510. For example, the maximum shear stress may be along the wall of the blood cell. Wall shear stress on blood cells is considered a major source of mechanical damage to blood cells causing hemolysis of the blood cells. In some embodiments, the insert portion 534 having the continuous non-linear channel 536 may facilitate increased flow resistance within the vascular access system 100 to distribute the pressure differential and reduce shear stress experienced by the red blood cells of the blood 15.

According to various embodiments of the present disclosure, the insert portion 534 may further include an inner surface 540 defining a lumen 542 of the insert portion 534. As depicted in FIG. 6B, with continued reference to FIG. 1C, the lumen 542 may form an internal flowpath along which a fluid 17 may flow from the proximal connector 530 into the catheter assembly 50 via the distal connector 512 and an extension tubing 60, for example. In some embodiments, the fluid flowing from the proximal connector 530 into the catheter assembly 50 via the distal connector 512 may be an IV fluid. In some embodiments, the internal flowpath in which the fluid flows from the proximal connector 530 into the catheter assembly 50 via the distal connector 512 may be a linear flowpath.

In accordance with some embodiments of the present disclosure, the flow restriction device 510 may further include a check valve 550 disposed in the lumen 542 of the insert portion 534. For example, the check valve 550 may be disposed at a proximal end of the insert portion 534. The check valve may selectively couple the fluid collection device 40 with the lumen 542 of the insert portion 534 with the distal connector 512. For example, the check valve 550 may be a normally closed valve such that the check valve 550 may prevent the fluid 15 (e.g., blood) from flowing from the distal connector 512 into the proximal connector 530 via the lumen 542 of the insert portion 534. The check valve 550 may however have a configuration to allow the fluid 17 (e.g., IV fluid) to flow from the proximal connector 530 into the distal connector 512 via the lumen 542 of the insert portion 534. For example, in some embodiments, the check valve 550 may be a check valve having a normally closed slit that is configured to open when subject to fluid pressure in the proximal-to-distal direction. Accordingly, when the fluid (e.g., IV fluid) is infused into the flow restriction device 510, the slit may open and allow the fluid to flow from the proximal connector 530 into the distal connector 512, and ultimately to the catheter assembly 50. When the fluid (e.g., blood) is being drawn from the catheter assembly 50 into the fluid collection device 40, the normally closed slit may stay closed and thereby prevent the fluid flowing from the distal connector 512 to the proximal connector 530 from entering the fluid collection device 40 via the lumen 542 of the insert portion 534. Accordingly, the blood sample 34 containing blood cells may be forced to flow through the curved or spiral continuous non-linear channel 536 in order to arrive at the blood collection device 40, while the IV fluid 17 may flow to the catheter assembly 50 through the internal flowpath defined by the lumen 542.

In some embodiments, the continuous non-linear channel 536 may have a first diameter D3, and the lumen 542 of the insert portion 534 may have a second diameter D4, and the first diameter D3 may be smaller than the second diameter D4. The continuous non-linear channel 536 can be formed by any of a length, a diameter, and a cross-sectional area as described above with reference to an optimized fluid pathway.

As a non-limiting example, in some embodiments, first diameter D3 may range from about 0.02 inches to 0.03 inches, in some instances range from about 0.022 inches to 0.028 inches, more typically from about 0.024 inches to 0.026 inches, and in some embodiments approximately 0.025 inches. Though recited in terms of certain ranges, it will be understood that all ranges from the lowest of the lower limits to the highest of the upper limits are included, including all intermediate ranges or specific angles, within this full range or any specifically recited range. However, the various embodiments of the present disclosure are not limited to the aforementioned configuration.

The aforementioned configuration is advantageous in that due to the minimized diameter or size of the fluid pathway defined by continuous non-linear channel 536, the fluid pathway defined by continuous non-linear channel 536 may thereby provide increased flow resistance and decreased blood flow rate within the flow resistance device 510 in contrast to the internal flowpath defined by the lumen 542 of the insert portion 534. Accordingly, a risk of hemolysis during blood collection may advantageously be reduced.

The aforementioned configuration with the second diameter D4 being larger than the first diameter D3 may further be advantageous in further allowing an unrestricted and increased amount of the IV fluid 17 to flow to the patient via the larger diameter internal flowpath defined by the lumen 542 versus flowing only in the smaller (minimized) diameter fluid pathway defined by continuous non-linear channel 536. Accordingly, the IV fluid 17 may flow in a second unrestricted (less flow resistance) direction (proximal to distal) opposite from the first direction (distal to proximal) in which the blood sample 15 having blood cells flows.

In operation, during blood collection or withdrawal from the patients, the blood 15 may flow from the patient's vein into the catheter assembly 50, through the extension tubing 60, enter the distal connector 512 of the flow restriction device 510 via a lumen 519 of the distal connector 512. Due to the presence of the check valve 550 in the lumen 542 of the insert portion 534, the blood 15 may be forced to flow around the outer surface of the insert portion 534 into the continuous non-linear channel 536, and exit the flow restriction device 510 into the blood collection device 40. Accordingly, during blood collection or withdrawal from the patient, the blood 15 may flow into the blood collection device 40 via the continuous non-linear channel 536 having minimal diameter. The flow restriction device 510 of the various embodiments described herein is advantageous over currently existing blood collection systems. For example, during blood draw with currently existing blood draw devices, blood cells may experience shear stress as they flow from the distal end to the proximal end of the blood collection systems. The maximum shear stress may be along the wall of the blood cell, often referred to as wall shear stress. Wall shear stress on blood cells is considered a major source of mechanical damage to blood cells causing hemolysis of the blood cells. In some embodiments, the continuous non-linear channel 536 having minimal diameter may facilitate increased flow resistance within the vascular access system 100 to distribute the pressure differential and reduce shear stress experienced by the red blood cells of the blood 15. For example, the minimized diameter of the continuous non-linear channel 536 may provide increased resistance to flow of the blood 15 and thereby decrease blood flow rate within the flow resistance device 510. Since the decreased blood flow rate causes a reduction in shear stress experienced by the red blood cells of the blood 15, a risk of hemolysis during blood collection may advantageously be reduced.

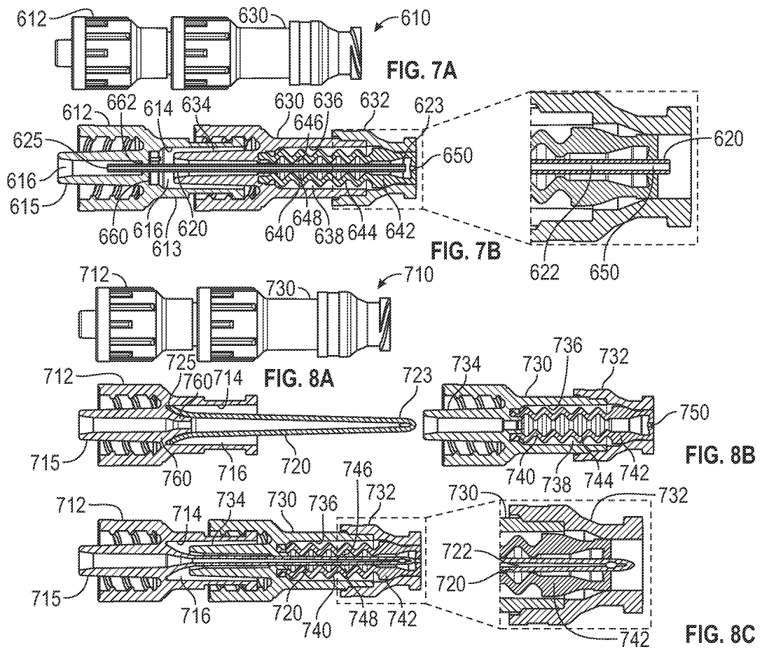

FIG. 7A illustrates a perspective view of a flow restriction device, in accordance with some embodiments of the present disclosure. FIG. 7B illustrates a cross-sectional view of the flow restriction device of FIG. 7A, in accordance with some embodiments of the present disclosure. As illustrated in FIGS. 7A and 7B, with continued reference to FIGS. 1A and 1B, a flow restriction device 610 may include a first connector 630 having a female luer portion 632 at a proximal end, a male luer portion 634 at distal end, an internal surface 636 defining an inner lumen 638 of the first connector 630, and a compressible valve member 640 mounted in the inner lumen 638. The first connector 630 may be configured to couple to a fluid collection device 40 (illustrated in FIGS. 1A and 1B). For example, in some embodiments, the first connector 630 may be a needleless connector including the female luer portion 632 at the proximal end, the male luer portion 634 at the distal end and the compressible valve member 640 mounted in the first connector, extending longitudinally in the inner lumen 638 of the needleless connector. In some embodiments, the compressible valve member 640 may include a head portion 642 having a slot, slit, or other similar form of cut 650 at a proximal end of the head portion 642, and a body portion 644 extending distally from the head portion 642. The body portion 644 may be configured to elastically compress and expand based on application and removal of an axial force. For example, in some embodiments, the body portion 644 may have an accordion or spring shape. The head portion 642 may be in the form of a split-septum. For example, as depicted, a proximal end of the head portion 642 may include the slot, slit, or other similar form of cut 650. In some embodiments, the valve member 640 may include an inner surface 646 defining an internal chamber 648 of the valve member 640.

In accordance with various embodiments of the present disclosure, flow restriction device 610 may further include a second connector 612 coupled to the male luer portion 634 of the first connector 630 and configured to couple to a catheter assembly 50. As depicted, the second connector 612 may have an internal surface 614 defining an inner lumen 616 of the second connector 612. In some embodiments, the second connector 612 may further include a support portion 660 extending radially inward from the internal surface 614 into the inner lumen 616 of the second connector 612. The support portion 660 may include a mounting aperture 662.

In some embodiments, a cannula 620 may be mounted in the inner lumen 616 of the second connector 612. In particular, the cannula 620 may be mounted in the mounting aperture 662 to fluidly communicate the second connector 612 with the fluid collection device 40. As depicted, the cannula 620 may extend from the inner lumen 616, through the male luer portion 634 of the first connector, and into the female luer portion 632 of the first connector 630. In some embodiments, the compressible valve member 640 may be mounted surrounding at least a portion of the cannula 620. For example, in some embodiments, a proximal portion of the cannula 620 may extend into the internal chamber 648 of the of the valve member 640. In particular, in some embodiments, the proximal portion of the cannula 620 may extend into the head portion 642 of the compressible valve member 640. In some embodiments, the cannula 620 may be press-fit in the support portion 660. However, the various embodiments of the present disclosure are not limited to the aforementioned configuration. In some embodiments, the cannula 620 may be fastened, attached, or otherwise coupled by any other suitable joining means.

In some embodiments, the cannula 620 may have a proximal end 623, a distal end 625, and a lumen 622 extending therethrough. The cannula 620 may extend from the lumen 616 of the second connector 612 into the internal chamber 648 of the compressible valve member 640 which is disposed in the inner lumen 638 of the first connector 630. The lumen 622 of cannula 620 may define a flowpath or micro-channel along which a fluid may flow from the second connector 612 into the fluid collection device 40. As depicted, the cannula 620 may be mounted in the mounting aperture 662 of the second connector support portion 660 and may fluidly couple the blood collection device 40 with the catheter assembly 50. For example, in some embodiments, a leg 72 of the Y-adapter 70 may be coupled to the flow restriction device 610. The leg 72 of Y-adapter 70 may include a lumen into which the distal end 615 of the second connector 612 having the cannula 620 mounted therein may be coupled. The Y-adapter 70 may fluidly communicate the flow restriction device 610, and cannula 620 mounted therein, with the catheter assembly 50, for example, via the extension tubing 60. Accordingly, the lumen 622 of the cannula 620 may define a linear fluid pathway with a reduced, small, or micro-sized diameter (as discussed below) through which fluid entering the flow restriction device 610 from the catheter assembly, may flow through the flow restriction device 610 for collection in the fluid collection device 40. For example, where blood is being withdrawn or collected from a patient, the medical fluid 15 may be blood, and the fluid collection device 40 may be a blood collection device. In some embodiments, the blood collection device may be a luer lock access device (LLAD). Accordingly, during blood collection or withdrawal from the patient, the blood sample 15 may flow from the distal end 615 of the second connector 612 into the LLAD 40 via the flowpath or micro-channel defined by the lumen 622.

In some embodiments, the cannula 620 may be an elongate, thin tube with the lumen having a small, reduced or micro-sized diameter. For example, in some embodiments, the lumen 622 of the cannula 620, that defines the flowpath or micro-channel along which fluid may flow from the distal end 615 into the fluid collection device 40 via cannula 620. The cannula 620 can define a lumen 622 formed by any of a length, a diameter, and a cross-sectional area as described above with reference to an optimized fluid pathway.

In order to draw fluid (e.g., blood) from the catheter assembly 50, the fluid collection device 40 may be inserted into and coupled to the female luer portion 632 of the first connector 630. In the coupled configuration of the first connector 630 and the fluid collection device 40, the compressible valve member 640 may be compressed distally by the fluid collection device 40 to fluidly communicate the proximal end 623 of cannula 620 and the fluid collection device 40. For example, as a male luer portion of the blood collection device 40 is inserted into the female luer portion 632 of the first connector 630, the male luer portion of the blood collection device 40 may move the head portion 642 of the valve member 640 and cause the valve member 640 to compress in the distal direction. As the head portion is displaced distally, the proximal end 623 of the cannula 620 may be exposed to the exterior of the valve member 640 via the slot 650. The proximal end 623 of the cannula 620 may thus be fluidly coupled to the male luer portion of the blood collection device 40 via the slot of the compressible valve member.

In operation, during blood collection or withdrawal from the patient, the blood 15 may flow from the patient's vein into the catheter assembly 50, through the extension tubing 60, and enter the distal end 615 of the second connector 612. Due to the presence of the cannula 620 in the lumen 616 of the second connector, the blood 15 may be forced to flow into and through the flowpath or micro-channel defined by the lumen 622, and exit the flow restriction device 610 into the blood collection device 40. Accordingly, during blood collection or withdrawal from the patient, the blood 15 may flow into the blood collection device 40 via the flowpath or micro-channel defined by the lumen 622 having minimal diameter. The flow restriction device 610 of the various embodiments described herein is advantageous over currently existing blood collection systems. For example, during blood draw with currently existing blood draw devices, blood cells may experience shear stress as they flow from the distal end to the proximal end of the blood collection systems. The maximum shear stress may be along the wall of the blood cell, often referred to as wall shear stress. Wall shear stress on blood cells is considered a major source of mechanical damage to blood cells causing hemolysis of the blood cells. In some embodiments, the continuous flowpath or micro-channel defined by the lumen 622 having minimal diameter may facilitate increased flow resistance within the vascular access system 100 to distribute the pressure differential and reduce shear stress experienced by the red blood cells of the blood 15. For example, the minimized diameter of the flowpath or micro-channel defined by the lumen 622 may provide increased resistance to flow of the blood 15 and thereby decrease blood flow rate within the flow resistance device 610. Since the decreased blood flow rate causes a reduction in shear stress experienced by the red blood cells of the blood 15, a risk of hemolysis during blood collection may advantageously be reduced.

FIG. 8A illustrates a perspective view of a flow restriction device 710, in accordance with some embodiments of the present disclosure. FIG. 8B illustrates a cross-sectional view of first and second connectors 730 and 712 of the flow restriction device of FIG. 8A, in accordance with some embodiments of the present disclosure. FIG. 8C illustrates a cross-sectional view of the flow restriction device 710 of FIG. 8A, in accordance with some embodiments of the present disclosure. As illustrated in FIGS. 8A and 8B, with continued reference to FIGS. 1A and 1B, a flow restriction device 710 may include a first connector 730 having a female luer portion 732 at a proximal end, a male luer portion 734 at distal end, an internal surface 736 defining an inner lumen 738 of the first connector 730, and a compressible valve member 740 mounted in the inner lumen 738. The first connector 730 may be configured to couple to a fluid collection device 40 (illustrated in FIGS. 1A and 1B). For example, in some embodiments, the first connector 730 may be a needleless connector including the female luer portion 732 at the proximal end, the male luer portion 734 at the distal end and the compressible valve member 740 mounted in the first connector 730, extending longitudinally in the inner lumen 738 of the needleless connector. In some embodiments, the compressible valve member 740 may include a head portion 742 having a slot, slit, or other similar form of cut 750 at a proximal end of the head portion 742, and a body portion 744 extending distally from the head portion 742. The body portion 744 may be configured to elastically compress and expand based on application and removal of an axial force. For example, in some embodiments, the body portion 744 may have an accordion or spring shape. The head portion 742 may be in the form of a split-septum. For example, as depicted, a proximal end of the head portion 742 may include the slot, slit, or other similar form of cut 750. In some embodiments, the valve member 740 may include an inner surface 746 defining an internal chamber 748 of the valve member 740.

In accordance with various embodiments of the present disclosure, flow restriction device 710 may further include a second connector 712 coupled to the male luer portion 734 of the first connector 730 and configured to couple to a catheter assembly 50. As depicted, the second connector 712 may have an internal surface 714 defining an inner lumen 716 of the second connector 712. In some embodiments, the second connector 712 may further include a support portion 760 extending proximally from a proximal end of the male luer portion 734 of the second connector 712 into the inner lumen 716 of the female luer portion 732 of the second connector 712.

In some embodiments, a post 720 may be mounted in the inner lumen 716 of the second connector 712. In particular, the post 720 may be mounted on or sleeved over the support portion 760 to fluidly communicate the second connector 712 with the fluid collection device 40. As depicted, the post 720 may extend from the inner lumen 716, through the male luer portion 734 of the first connector, and into the female luer portion 732 of the first connector 730. In some embodiments, the compressible valve member 740 may be mounted surrounding at least a portion of the post 720. For example, in some embodiments, a proximal portion of the post 720 may extend into the internal chamber 748 of the of the valve member 740. In particular, in some embodiments, the proximal portion of the post 720 may extend into the head portion 742 of the compressible valve member 740. In some embodiments, the post 720 may be press-fit onto the support portion 760. However, the various embodiments of the present disclosure are not limited to the aforementioned configuration. In some embodiments, the post 720 may be fastened, attached, or otherwise coupled to the support portion 760 by any other suitable joining means.

In some embodiments, the post 720 may be in the form of an elongate tube having a proximal end 723, a distal end 725, and a lumen 722 extending therethrough. In some embodiments, the post 720 may have a shape that tapers from the distal end 725 to the proximal end 723 of the post 720. Accordingly, a shape or profile of the lumen 722 may also taper from the distal end 725 to the proximal end 723 of the post 720. As depicted, the post 720 may extend from the lumen 716 of the second connector 712 into the internal chamber 748 of the compressible valve member 740 which is disposed in the inner lumen 738 of the first connector 730. The lumen 722 of post 720 may define a flowpath or micro-channel along which a fluid may flow from the second connector 712 into the fluid collection device 40. As depicted, the post 720 may be mounted in the mounting aperture 762 of the second connector support portion 760 and may fluidly couple the blood collection device 40 with the catheter assembly 50. For example, in some embodiments, a leg 72 of the Y-adapter 70 may be coupled to the flow restriction device 710. The leg 72 of Y-adapter 70 may include a lumen into which the distal end 715 of the second connector 712 having the post 720 mounted therein may be coupled. The Y-adapter 70 may fluidly communicate the flow restriction device 710, and post 720 mounted therein, with the catheter assembly 50, for example, via the extension tubing 60. Accordingly, the lumen 722 of the post 720 may define a linear fluid pathway with a reduced, small, or micro-sized diameter (as discussed below) through which fluid entering the flow restriction device 710 from the catheter assembly 50, may flow through the flow restriction device 710 for collection in the fluid collection device 40. For example, where blood is being withdrawn or collected from a patient, the medical fluid 15 may be blood, and the fluid collection device 40 may be a blood collection device. In some embodiments, the blood collection device 40 may be a luer lock access device (LLAD). Accordingly, during blood collection or withdrawal from the patient, the blood sample 15 may flow from the distal end 715 of the second connector 712 into the LLAD 40 via the flowpath or micro-channel defined by the lumen 722.

As described above, the post 720 may be an elongate, thin tube with the lumen 722 having a small, reduced or micro-sized diameter. For example, in some embodiments, the lumen 722 of the post 720, that defines the flowpath or micro-channel along which fluid may flow from the distal end 715 into the fluid collection device 40 via post 720. The post 720 can define a lumen 722 formed by any of a length, a diameter, and a cross-sectional area as described above with reference to an optimized fluid pathway.

In order to draw fluid (e.g., blood) from the catheter assembly 50, the fluid collection device 40 may be inserted into and coupled to the female luer portion 732 of the first connector 730. In the coupled configuration of the first connector 730 and the fluid collection device 40, the compressible valve member 740 may be compressed distally by the fluid collection device 40 to fluidly communicate the proximal end 723 of post 720 and the fluid collection device 40. For example, as a male luer portion of the blood collection device 40 is inserted into the female luer portion 732 of the first connector 730, the male luer portion of the blood collection device 40 may move the head portion 742 of the valve member 740 and cause the valve member 740 to compress in the distal direction. As the head portion 642 is displaced distally, the proximal end 723 of the post 720 may be exposed to the exterior of the valve member 740 via the slot 750. The proximal end 723 of the post 720 may thus be fluidly coupled to the male luer portion of the blood collection device 40 via the slot 750 of the compressible valve member 740.

In operation, during blood collection or withdrawal from the patient, the blood 15 may flow from the patient's vein into the catheter assembly 50, through the extension tubing 60, and enter the distal end 715 of the second connector 712. Due to the presence of the post 720 in the lumen 716 of the second connector, the blood 15 may be forced to flow into and through the flowpath or micro-channel defined by the lumen 722, and exit the flow restriction device 710 into the blood collection device 40. Accordingly, during blood collection or withdrawal from the patient, the blood 15 may flow into the blood collection device 40 via the flowpath or micro-channel defined by the lumen 722 having minimal diameter. The flow restriction device 710 of the various embodiments described herein is advantageous over currently existing blood collection systems. For example, during blood draw with currently existing blood draw devices, blood cells may experience shear stress as they flow from the distal end to the proximal end of the blood collection systems. The maximum shear stress may be along the wall of the blood cell, often referred to as wall shear stress. Wall shear stress on blood cells is considered a major source of mechanical damage to blood cells causing hemolysis of the blood cells. In some embodiments, the continuous flowpath or micro-channel defined by the lumen 722 having minimal diameter may facilitate increased flow resistance within the vascular access system 100 to distribute the pressure differential and reduce shear stress experienced by the red blood cells of the blood 15. For example, the minimized diameter of the flowpath or micro-channel defined by the lumen 722 may provide increased resistance to flow of the blood 15 and thereby decrease blood flow rate within the flow resistance device 710. Since the decreased blood flow rate causes a reduction in shear stress experienced by the red blood cells of the blood 15, a risk of hemolysis during blood collection may advantageously be reduced.

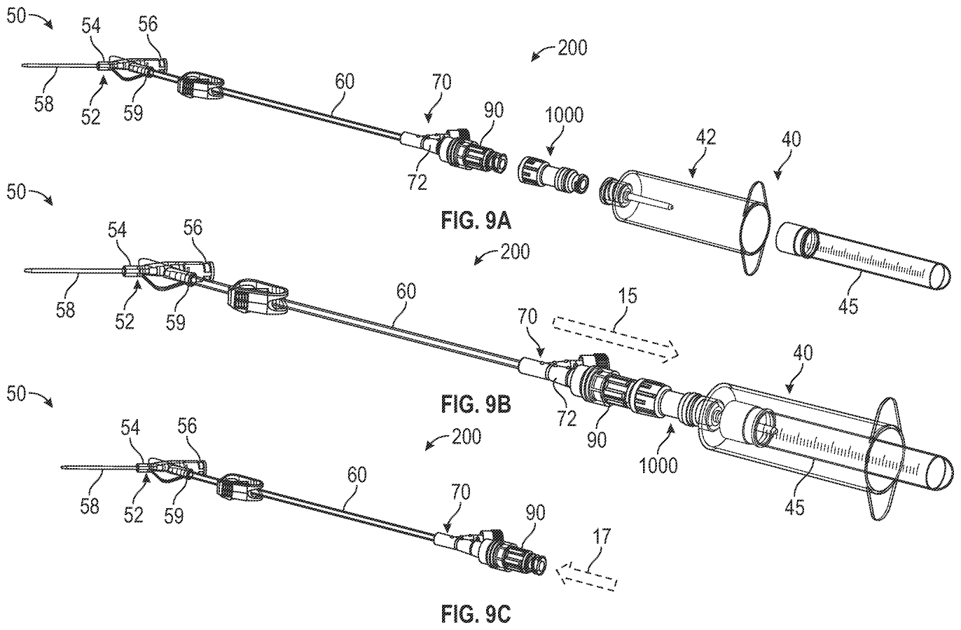

FIG. 9A illustrates an exploded view of a vascular access device including a peripheral intravenous catheter (PIVC) assembly having a flow restriction device and a fluid collection device, in accordance with some embodiments of the present disclosure. FIG. 9B is an operational illustration of a vascular access device including a peripheral intravenous catheter (PIVC) assembly having a flow restriction device and a fluid collection device, in accordance with some embodiments of the present disclosure. FIG. 9C is an operational illustration of a vascular access device including a peripheral intravenous catheter (PIVC) assembly having a flow restriction device and a needleless connector, in accordance with some embodiments of the present disclosure. Referring now to FIGS. 9A-9C, flow restriction device 1000 is illustrated, according to some embodiments. The flow restriction device 1000 may be configured to reduce a likelihood of hemolysis during blood collection using a vascular access device 200. In some embodiments, the vascular access device 200 may include a catheter assembly (e.g., a PIVC) 50. In some embodiments, the flow restriction device 1000 may include a distal end, which may include a body or distal connector configured to couple to the catheter assembly 50. The distal connector may include a male luer connector, or another suitable connector.

In some embodiments, the catheter assembly 50 may include a catheter hub 52, which may include a distal end 54, a proximal end 56, and a lumen extending through the distal end and the proximal end. The catheter assembly 50 may further include a catheter 58, which may be secured within the catheter hub 52 and may extend distally from the distal end 54 of the catheter hub 52. In some embodiments, the catheter may be a peripheral intravenous catheter (PIVC).

In some embodiments, the catheter assembly 50 may include or correspond to any suitable catheter assembly 50. In some embodiments, the catheter assembly 50 may be integrated and include an extension tube 60, which may extend from and be integrated with a side port 59 of the catheter hub 52. A non-limiting example of an integrated catheter assembly is the BD NEXIVA™ Closed IV Catheter system, available from Becton Dickinson and Company. In some embodiments, a proximal end of the extension tube 60 may be coupled to an adapter 70, such as, for example, a Y-adapter or single port luer adapter. In some embodiments, the distal connector of flow restriction device 1000 may be configured to couple to the Y-adapter 70.

In some embodiments, the catheter assembly 50 may be non-integrated and may not include the extension tube 60. In these and other embodiments, the flow restriction device 1000 may be configured to couple to the proximal end 56 of the catheter hub 52 or another suitable portion of the catheter assembly 50. In some embodiments, the catheter assembly 50 may be coupled to a removable extension tube 60. In some embodiments, the flow restriction device 1000 may be coupled directly to the catheter adapter, eliminating the extension tube and providing a compact catheter system.

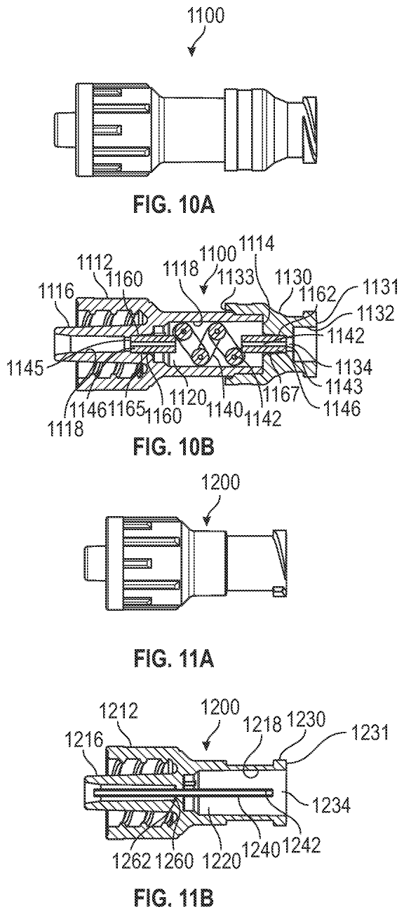

FIG. 10A illustrates a perspective view of a flow restriction device 1100, in accordance with some embodiments of the present disclosure. FIG. 10B illustrates a cross-sectional view of the flow restriction device 1100 of FIG. 10A, in accordance with some embodiments of the present disclosure. As illustrated in FIGS. 10A and 10B, with continued reference to FIGS. 9A and 9B, in some embodiments, the flow restriction device 1100 may include a male luer connector portion 1112 configured to couple to the catheter assembly 50. The male luer connector portion 1112 may have a proximal end 1114, a distal end 1116, and an internal surface 1118 defining an inner lumen 1120 of the male luer connector portion 1112. As depicted, the male luer connector portion 1112 may further include a support portion 1160 disposed between the proximal and distal ends 1114 and 1116. The support portion 1160 may include a mounting aperture 1165 extending therethrough.

In some embodiments, the flow restriction device 1100 may further include a female luer connector portion 1130 coupled to the proximal end 1114 of the male luer connection portion 1112. The female luer connector portion 1130 may be configured to couple to fluid collection device 40 (e.g., a blood collection device). For example, the female luer connector portion 1130 may be integrated with the blood collection device 40 or monolithically formed with the blood collection device 40 as a single unit. As another example, the female luer connector portion 1130 may be in the form of a female luer connector or another suitable connector, which may be coupled with a male luer portion of the blood collection device 40. The female luer connector portion 1130 may have a proximal end 1131, a distal end 1133, and an internal surface 1132 defining a lumen 1134 extending therethrough for coupling to the male luer portion of the blood collection device 40. As depicted, the female luer connector portion 1130 may further include a support portion 1162 disposed between the proximal and distal ends 1131 and 1133. The support portion 1162 may include a mounting aperture 1167 extending therethrough.

In accordance with various embodiments of the present disclosure, the flow restriction device 1110 may further include a tubing 1140 mounted in at least one of the lumens 1120 and 1134 of the male luer connector portion 1112 and the female luer connector portion 1130. For example, in some embodiments, the tubing 1140 may be mounted in at least one of the support portions 1160 and 1162. As depicted, the tubing 1140 may be mounted or otherwise supported in both the support portions 1160 and 1162 and may extend from the support portion 1160 of the male luer connector portion 1112 into the female luer connector portion 1130. The tubing 1140 may have a proximal end 1143, a distal end 1145, and a lumen 1142 extending therethrough. The lumen 1142 may define a flowpath or micro-channel along which a fluid may flow from the male luer connector portion 1112 into the fluid collection device 40 via the female luer connector portion 1130. As depicted, the tubing 1140 may fluidly communicate the catheter assembly 50 with the fluid collection device 40 via the flow restriction device 1100. For example, in some embodiments, a leg 72 of the Y-adapter 70 may be coupled to the flow restriction device 1110. The leg 72 of Y-adapter 70 may include a lumen into which the distal end 1116 of the male luer connector portion 1112 having the tubing 1140 mounted therein may be coupled. The Y-adapter 70 may fluidly communicate the flow restriction device 1100, and tubing 1140 mounted therein, with the catheter assembly 50, for example, via the extension tubing 60. Accordingly, the lumen 1142 of the tubing 1140 may define a fluid pathway with a reduced, small, or micro-sized diameter (as discussed below) through which fluid entering the flow restriction device 1100 from the catheter assembly 50, may flow through the flow restriction device 1100 for collection in the fluid collection device 40. For example, where blood is being withdrawn or collected from a patient, the medical fluid 15 may be blood, and the fluid collection device 40 may be a blood collection device. In some embodiments, the blood collection device may be a luer lock access device (LLAD). Accordingly, during blood collection or withdrawal from the patients, the blood sample 15 may flow from the distal end 1116 of the male luer connector portion 1112 into the LLAD 40 via the flowpath or micro-channel defined by the lumen 1142.

In some embodiments, the flowpath or micro-channel defined by the lumen 1142 may have a spiral shape, coil shape, S-shape, or other suitable non-linear, winding shape. The aforementioned configuration is advantageous in that the spiral shape, coil shape, S-shape, or other suitable non-linear, winding shape of the flowpath or micro-channel defined by the lumen 1142—by virtue of its winding nature—may increase a length of the fluid pathway defined by the flowpath or micro-channel defined by the lumen 1142 as compared to a linear micro-channel.

Where the medical fluid is blood being withdrawn or collected from a patient, the medical fluid may be a blood sample, and the fluid collection device 40 may be a blood collection device. In some embodiments, the blood collection device may be a luer lock access device (LLAD). Where the medical fluid is blood being withdrawn from a patient, blood cells may experience wall shear stress as they flow from the male luer connector portion 1112 to the female luer connector portion 1130 of the flow restriction device 1100. Wall shear stress on blood cells is considered a major source of mechanical damage to blood cells causing hemolysis of the blood cells. In some embodiments, the flow restriction device 1110 having the non-linear, winding flowpath or micro-channel defined by the lumen 1142 may facilitate increased flow resistance within the vascular access system 1100 to distribute the pressure differential and reduce shear stress experienced by the red blood cells of the blood 15. In some embodiments, the tubing 1140

In some embodiments, the tubing 1140 may be a thin tube with the lumen having a small, reduced or micro-sized diameter. For example, in some embodiments, the lumen 1142 of the tubing 1140, that defines the first flowpath or micro-channel along which fluid may flow from the male luer connector portion 1112 into the fluid collection device 40 via the female luer connector portion 1130 The tubing 1140 can define a lumen 1142 formed by any of a length, a diameter, and a cross-sectional area as described above with reference to an optimized fluid pathway.

As previously described, when the fluid 15 is blood being withdrawn from a patient, blood cells may experience wall shear stress as they flow from the catheter assembly 50 into the blood collection device 40. For example, the maximum shear stress may be along the wall of the blood cell, often referred to as wall shear stress. In some embodiments, the lumen 1142 of the tubing 1140 having a reduced or micro-sized diameter may facilitate increased flow resistance within the vascular access system 1100 to distribute the pressure differential and reduce shear stress experienced by the red blood cells of the blood 15. The minimized diameter of the first fluid pathway or micro-channel defined by the lumen 1142 of the tubing 1140 may provide increased resistance to flow of the blood 15 and thereby decrease blood flow rate within the flow resistance device 1110. Since the decreased blood flow rate causes a reduction in shear stress experienced by the red blood cells of the blood 15, a risk of hemolysis during blood collection may advantageously be reduced.

In some embodiments, at least a portion of the tubing 1140 may be a linear tubing 1146. For example, in some embodiments, the tubing 1140 may include at least one linear portion 1146 at a proximal end and a distal end thereof. As depicted, the proximal end 1143 of the tubing 1140 and the distal end 1145 of the tubing 1140 may be formed of the linear tubing 1146. The linear tubing 1146 at the proximal end may be mounted in the mounting aperture 1167 of support portion 1162 and the linear tubing 1146 at the distal end may be mounted in the mounting aperture 1165 of support portion 1160. In some embodiments, linear portions 1146 of the tubing 1140 may be in the form of a thin tube with a lumen having a small, reduced or micro-sized diameter. In some embodiments, the lumen 1142 of the tubing 1140 may have a same diameter in the linear portions 1146 as in the spiral shape, coil shape, S-shape, or other suitable non-linear, winding shape flowpath or micro-channel described above. Any of the tubing 1140 and the linear portions 1146 can define a lumen formed by any of a length, a diameter, and a cross-sectional area as described above with reference to an optimized fluid pathway.

As depicted, the spiral shape, coil shape, S-shape, or other suitable non-linear, winding shape flowpath or micro-channel may be interposed between the linear portions 1146 of the tubing 1140. Accordingly, during fluid draw, e.g., blood draw, the blood drawn from the catheter assembly 50 may travel from the distal end 1116 of the male luer connector portion 1112 into the micro-sized diameter lumen of the distal linear portion 1146, through the spiral shape, coil shape, S-shape, or other suitable non-linear, winding shape of flowpath or micro-channel of the lumen 1142, out of the micro-sized diameter lumen of the proximal linear portion 1146, and finally into the blood collection device 40.

FIG. 11A illustrates a perspective view of a flow restriction device 1200, in accordance with some embodiments of the present disclosure. FIG. 11B illustrates a cross-sectional view of the flow restriction device 1200 of FIG. 11A, in accordance with some embodiments of the present disclosure. The flow restriction device 1200 may be similar in structure to the flow restriction device 1100 with the exception that the flow restriction device 1200 may not include the female luer connector portion 1130. Instead, as depicted, the proximal end 1231 of the male luer connector portion 1212 may be open and include at least one thread 1230 on an outer surface thereof for coupling to the blood collection device 40. Additionally, the flow restriction device 1200 may differ from the flow restriction device 1100 in that tubing 1240 may be a linear tubing. For example, in some embodiments, the linear tubing may be in the form of a cannula 1240 mounted in the male luer connector portion 1212. As such, the male luer connector portion 1212 may further include a support portion 1260 the extending radially inward from the internal surface 1218 of the male luer connector portion 1212 into the lumen 1220 of the male luer connector portion 1212. As depicted, the support portion 1260 may include a mounting aperture 1262, and the cannula 1240 may be mounted in the mounting aperture 1262 for fluidly communicating the male luer connector portion 1212 with the fluid collection device 40. In some embodiments, the cannula 1240 may be press-fit in the support portion 1260. However, the various embodiments of the present disclosure are not limited to the aforementioned configuration, and the cannula may be attached by any other appropriate fastening means such as welding.

In some embodiments, the cannula 1240 may be an elongate, thin tube with the lumen having a small, reduced or micro-sized diameter. For example, in some embodiments, the lumen 1242 of the cannula 1240, that may define a flowpath or micro-channel along which fluid may flow from the distal end 1216 into the fluid collection device 40. The cannula 1240 can define a lumen 1242 formed by any of a length, a diameter, and a cross-sectional area as described above with reference to an optimized fluid pathway.

Accordingly, during blood collection or withdrawal from the patient, the blood 15 may flow into the blood collection device 40 via the flowpath or micro-channel defined by the lumen 1242 having minimal diameter. The flow restriction device 1210 of the various embodiments described herein is advantageous over currently existing blood collection systems. For example, during blood draw with currently existing blood draw devices, blood cells may experience wall shear stress as they flow from the distal end to the proximal end of the blood collection systems. Wall shear stress on blood cells is considered a major source of mechanical damage to blood cells causing hemolysis of the blood cells. The flowpath or micro-channel defined by the lumen 1242 having minimal diameter may facilitate increased flow resistance within the vascular access system to distribute the pressure differential and reduce shear stress experienced by the red blood cells of the blood 15. For example, the minimized diameter of the flowpath or micro-channel defined by the lumen 1242 may provide increased resistance to flow of the blood 15 and thereby decrease blood flow rate within the flow resistance device 1210. Since the decreased blood flow rate causes a reduction in shear stress experienced by the red blood cells of the blood 15, a risk of hemolysis during blood collection may advantageously be reduced.

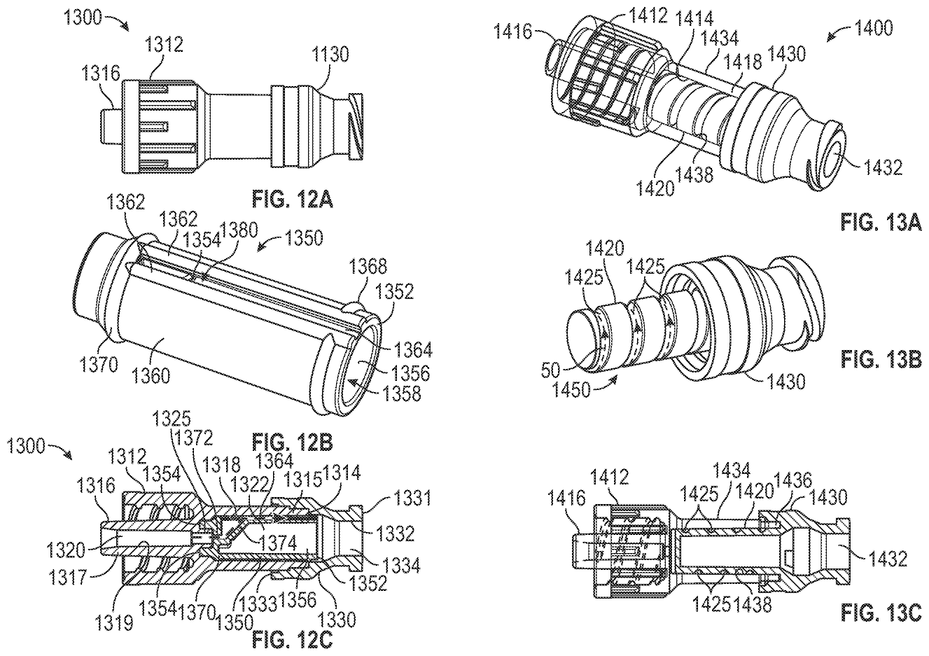

FIG. 12A illustrates a perspective view of a flow restriction device 1300, in accordance with some embodiments of the present disclosure. FIG. 12B illustrates a perspective view of an insert 1350 of the flow restriction device of FIG. 12A, in accordance with some embodiments of the present disclosure. FIG. 12C illustrates a cross-sectional view of the flow restriction device 1300 of FIG. 12A, in accordance with some embodiments of the present disclosure.

As illustrated in FIGS. 12A-12C, with continued reference to FIGS. 9A and 9B, in some embodiments, the flow restriction device 1300 may include a distal connector 1312 configured to couple to the catheter assembly 50. The distal connector 1312 may have a proximal end 1314 including a first connection portion 1315, and a distal end 1316 including a second connection portion 1317. The first connection portion 1315 may have an inner surface 1318 defining a lumen 1322 therethrough, and the second connection portion 1317 may have an inner surface 1319 defining a lumen 1320 therethrough. As depicted, the distal connector 1312 may further include a support portion 1325 disposed between the proximal end 1314 and the distal end 1317. The support portion 1325 may include mounting apertures 1354 recessed therein.

In some embodiments, the flow restriction device 1300 may further include a proximal connector 1330 coupled to the proximal end 1314 of the distal connector 1312. The proximal connector 1330 may be configured to couple to fluid collection device 40 (e.g., a blood collection device). For example, the proximal connector 1330 may be integrated with the blood collection device 40 or monolithically formed with the blood collection device 40 as a single unit. As another example, the proximal connector 1330 may be in the form of a female luer connector or another suitable connector, which may be coupled with a male luer portion of the blood collection device 40. The proximal connector 1330 may have a proximal end 1331, a distal end 1333, and an internal surface 1332 defining a lumen 1334 extending therethrough for coupling to the male luer portion of the blood collection device 40.

In accordance with various embodiments of the present disclosure, the flow restriction device 1300 may further include an insert 1350 mounted in the lumen 1322 of the first connection portion 1315 and interposed between the proximal connector 1330 and the distal connector 1312. As depicted, the insert 1350 may have an outer surface 1360 with a groove 1364 recessed therein. The groove 1364 may extend at least partially along a length of the outer surface 1360 and may be fluidly coupled to the lumen 1320 of the of the second connection portion 1317 and the lumen 1322 of the first connection portion 1315. In some embodiments, the groove 1364 may be a linear groove recessed in the outer surface 1360. For example, in some embodiments, the linear groove 1364 may be recessed in an upper portion of the outer surface 1360.

As depicted in FIG. 12C with continued reference to FIG. 12B, the insert 1350 may include a first channel section 1372 fluidly coupled to a proximal end of the lumen 1320 of the second connection portion 1317, and a second channel section 1374 extending from the first channel section to the linear groove 1364. The first and second channel sections 1372 and 1374 and the linear groove 1364 may together define the fluid channel 1380 along which the fluid flows from the distal connector 1312 into the fluid collection device 40 via the proximal connector 1330. In some embodiments, the first channel section 1372 and the linear groove 1364 may be offset from each other in position. For example, as depicted in FIG. 12C, in some embodiments the first channel section 1372 may be positioned at a first height and the linear groove 1364 may be positioned at a second height. In some embodiments, the second height may be greater than the first height, i.e., the linear groove 1364 may be positioned above or higher than the first channel section 1372.

In some embodiments, the second channel section 1374 may be in the form of a ramped surface which connects or otherwise fluidly couples the first channel section 1372 to the linear groove 1364. For example, as depicted, the second channel section 1374 may be interposed between and connect the first channel section 1372 with the linear groove 1364.

In some embodiments, the first connection portion 1315 may form an outer component for coupling with the insert 1350, and the insert 1350 may form an inner component having the fluid channel 1380 (defined by the first and second channel sections 1372 and 1374 and the linear groove 1364) which may be coupled in the lumen 1322 of the first connection portion 1315. Accordingly, the linear groove 1364 may be surrounded, encased, or otherwise enveloped in the lumen of the first connection portion 1315. The linear groove 1364 when encased, inserted or otherwise enveloped in the lumen 1322 by the inner surface 1318 of the first connection portion 1315 may define a micro-channel fluid pathway through which fluid (e.g., blood) flows from the second connection portion 1317 to the first connection portion 1315 for collection in the fluid collection device 40.

In some embodiments, the flowpath or micro-channel along which fluid may flow from the second connection portion 1317 to the first connection portion 1315 for collection into the fluid collection device 40, may be defined by any of a length, a diameter, and a cross-sectional area as described above with reference to an optimized fluid pathway.

Accordingly, during blood collection or withdrawal from the patient, the blood 15 may flow into the blood collection device 40 via the flowpath or micro-channel defined by the encased groove 1364 having minimal diameter. The flow restriction device 1300 of the various embodiments described herein is advantageous over currently existing blood collection systems. For example, during blood draw with currently existing blood draw devices, blood cells may experience wall shear stress as they flow from the distal end to the proximal end of the blood collection systems. As previously described above, wall shear stress on blood cells is considered a major source of mechanical damage to blood cells causing hemolysis of the blood cells. The flowpath or micro-channel defined by the encased groove 1364 having minimal diameter may facilitate increased flow resistance within the vascular access system to distribute the pressure differential and reduce shear stress experienced by the red blood cells of the blood 15. For example, the minimized diameter of the flowpath or micro-channel defined by the encased groove 1364 may provide increased resistance to flow of the blood 15 and thereby decrease blood flow rate within the flow resistance device 1210. Since the decreased blood flow rate causes a reduction in shear stress experienced by the red blood cells of the blood 15, a risk of hemolysis during blood collection may advantageously be reduced.

According to various embodiments of the present disclosure, as depicted for example in FIG. 12B, the outer surface 1360 of the insert 1350 may include longitudinally extending ledges 1362 disposed on and extending at least partially along a length of the outer surface 1360. As illustrated, the ledges 1362 may be positioned at opposing edges of the linear groove 1364 for sealing the edges. In some embodiments, the longitudinally extending ledges 1362 may bound the groove 1364 such that fluid flowing through the continuous channel or groove 1364 may not escape the continuous channel or groove 1364 except at a distal end 1354 and a proximal end 1352 of the groove 1364. In some embodiments, the longitudinally extending ledges 1362 may be a seal element, which may include silicon, rubber, plastic, or another suitable material. Accordingly, the longitudinally extending ledges 1362 may prevent fluid from escaping the continuous channel or groove 1364 except at the distal end 1354 and the proximal end 1352 of the groove 1364.

According to various embodiments, the proximal end of the groove 1364 may be fluidly connected to the lumen 1334 of the proximal connector for the medical fluid to be drawn into the fluid collection device 40 which is coupled to the proximal connector. Similarly, in some embodiments, the distal end 1354 of the groove 1364 may be fluidly connected to the lumen 1320 of the second connection portion 1317 to receive the medical fluid from the catheter assembly 50. Where the medical fluid is blood being withdrawn or collected from a patient, the medical fluid may be a blood sample, and the fluid collection device 40 may be a blood collection device. In some embodiments, the blood collection device may be a luer lock access device (LLAD).

FIG. 13A illustrates a perspective view of a flow restriction device, in accordance with some embodiments of the present disclosure. FIG. 13B illustrates a perspective view of proximal connector of the flow restriction device of FIG. 13A, in accordance with some embodiments of the present disclosure. FIG. 13C illustrates a cross-sectional view of the flow restriction device of FIG. 13A, in accordance with some embodiments of the present disclosure.

As illustrated in FIGS. 13A-13C, with continued reference to FIGS. 9A and 9B, in some embodiments, the flow restriction device 1400 may include a distal connector 1412 configured to couple to a catheter assembly 50, and a proximal connector 1430 coupled to the distal connector 1412 and configured to couple to a fluid collection device 40. Accordingly, during draw of a fluid (e.g., blood draw), the fluid may flow from the catheter assembly 50 into the flow restriction device 1400 (e.g., via the extension tubing 60), and exit the flow restriction device 1400 into the blood collection device 40. In some embodiments, the distal connector 1412 may include a first connection portion 1434 at a proximal end thereof and a second connection portion 1416 at a distal end thereof. As depicted, the first connection portion 1434 may be in the form of a cylindrical body having an inner surface 1414 defining a lumen 1418 of the distal connector 1412. The lumen 1418 may be configured such that at least a portion of the proximal connector 1430 may be inserted, fitted, or otherwise coupled therein to fluidly couple the distal and proximal connectors 1412 and 1430. For example, in some embodiments the proximal connector 1430 may include an insert portion 1450 for inserting into the lumen 1418 of the first connection portion 1434. As depicted, an outer surface 1420 of the insert portion 1450 may include a continuous non-linear channel 1425 recessed therein. In the coupled or assembled configuration of the of the distal connector 1412 and the proximal connector 1430, the inner surface 1414 of the first connection 1434 portion may encase, surround, or otherwise envelope the outer surface 1420 of the insert portion 1450 such that the continuous non-linear channel 1425 and the inner surface 1414 of the first connection portion 1434 define a non-linear fluid pathway along which a fluid (e.g., blood) may flow from the distal connector 1412 into the fluid collection device 40 via the proximal connector 1430.

In some embodiments, the continuous non-linear channel 1425 may form a coil shape, an S-shape, or another suitable non-linear, winding shape. For example, the continuous non-linear channel 1425 may have a coil shape (which may include a spiral) recessed in the outer surface 1420 of the insert portion 1450. In some embodiments, the continuous non-linear channel 1425 may have an S-shape recessed in the outer surface 1420 of the insert portion 1450. The aforementioned configuration is advantageous in that the spiral, coil shape, S-shape, or other suitable non-linear, winding shape of the continuous non-linear channel 1425—by virtue of its wrapping around the outer surface 1420 of the insert portion 1450—may increase a length of the fluid pathway defined by the continuous non-linear channel 1425 through which the medical fluid flows.

In some embodiments, the continuous non-linear channel 1425 may have a small, reduced or micro-sized diameter. For example, in some embodiments, the continuous non-linear channel 1425 that defines the flowpath or micro-channel along which fluid may flow from the distal connector 1416 into the fluid collection device 40 via the proximal connector 1430. The continuous non-linear channel 1425 can define a flowpath or micro-channel formed by any of a length, a diameter, and a cross-sectional area as described above with reference to an optimized fluid pathway.

As previously described, when the fluid 15 is blood being withdrawn from a patient, blood cells may experience wall shear stress as they flow from the catheter assembly 50 into the blood collection device 40. For example, the maximum shear stress may be along the wall of the blood cell, often referred to as wall shear stress. The continuous non-linear channel 1425 having a reduced or micro-sized diameter may facilitate increased flow resistance within the vascular access system to distribute the pressure differential and reduce shear stress experienced by the red blood cells of the blood 15. The minimized diameter of the continuous non-linear channel 1425 may provide increased resistance to flow of the blood 15 and thereby decrease blood flow rate within the flow resistance device 1400. Since the decreased blood flow rate causes a reduction in shear stress experienced by the red blood cells of the blood 15, a risk of hemolysis during blood collection may advantageously be reduced. Where the medical fluid is blood being withdrawn or collected from a patient, the medical fluid may be a blood sample, and the fluid collection device 40 may be a blood collection device. In some embodiments, the blood collection device may be a luer lock access device (LLAD).

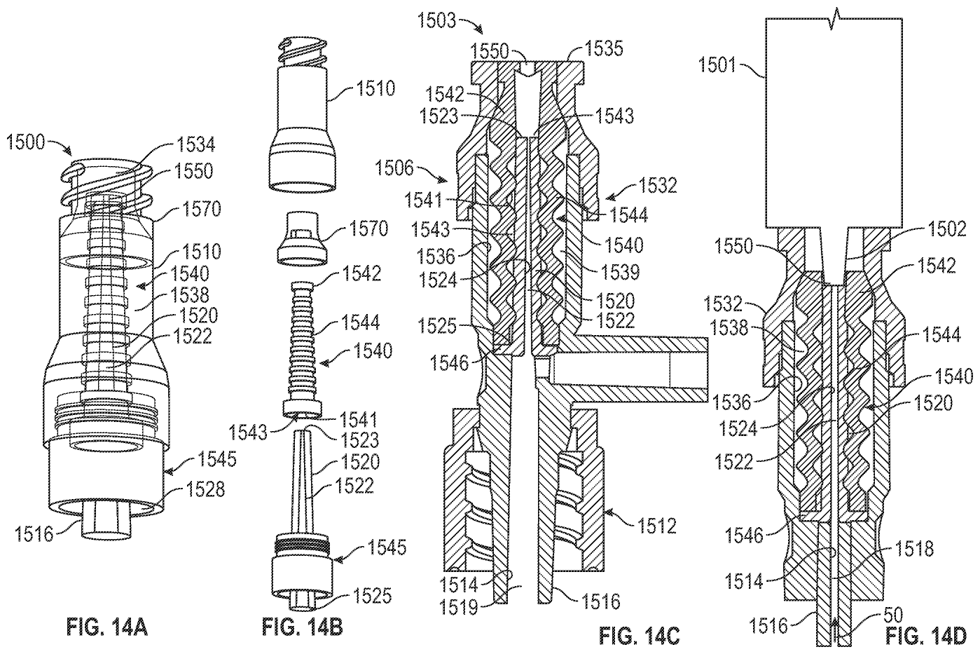

FIG. 14A illustrates a perspective view of a flow restriction device, in accordance with some embodiments of the present disclosure. FIG. 14B illustrates an exploded view of the flow restriction device of FIG. 14A, in accordance with some embodiments of the present disclosure. As illustrated in FIGS. 14A and 14B, with continued reference to FIGS. 9A and 9B, in some embodiments, the flow restriction device 1500 may include a connector 1505 having a body portion 1510 with a lumen 1538 and disposed at a proximal end, and a base portion 1545 having a lumen 1518 and disposed at distal end. The body portion 1510 may be configured to couple to a fluid collection device 40. In some embodiments, the flow restriction device 1500 may further include a compressible valve member 1540 mounted on the base portion 1545 and extending into the lumen 1538 of the body portion 1510. The compressible valve member 1540 may include an inner surface 1541 defining an internal chamber 1543 of the compressible valve 1540. As depicted, the compressible valve member 1540 may have a head portion 1542 including a slot 1550, and a body portion 1544 extending distally from the head portion 1542. In some embodiments, the body portion 1544 may have an accordion shape, or any other similar compressible or collapsible shape. In some embodiments, the head portion 1542 having the slot 1550 may be a split-septum head portion.

According to various embodiments of the present disclosure, the flow restriction device 1500 may further include a post 1520 having a lumen 1522 extending therethrough. The post 1520 may be mounted in the lumen 1518 of the base portion and extend into the internal chamber 1543 of the compressible valve member 1540. Accordingly, the compressible valve member 1540 may be mounted surrounding the post 1520. The post 1520 may be configured to be fluidly communicated with a catheter assembly 50. In some embodiments, the post 1520 may be press-fit into the lumen 1518 of the base portion 1545. However, the various embodiments of the present disclosure are not limited to the aforementioned configuration. In some embodiments, the post 1520 may be fastened, attached, or otherwise coupled in the lumen 1518 of the base portion 1545 by any other suitable joining means.

In some embodiments, the post 1520 may be in the form of an elongate tube having a proximal end 1523, a distal end 1525, and the lumen 1522 extending therethrough. In some embodiments, the post 1520 may have a shape that tapers from the distal end 1525 to the proximal end 1523 of the post 1520. Accordingly, a shape or profile of the lumen 1522 may also taper from the distal end 1525 to the proximal end 1523 of the post 1520. As depicted, the post 1520 may extend from the distal end 1523 into the internal chamber 1541 of the compressible valve member 1540 which is disposed in the inner lumen 1538 of the body portion 1510. The lumen 1522 of post 720 may define a flowpath or micro-channel along which a fluid may flow from the distal end 1525 into the fluid collection device 40. The post 1520 may be mounted in a mounting aperture of a support portion (not shown) of the base portion 1545 and may fluidly couple the blood collection device 40 with the catheter assembly 50. For example, with continued reference to FIGS. 9A and 9B, in some embodiments, a leg 72 of the Y-adapter 70 may be coupled to the flow restriction device 1500. The leg 72 of Y-adapter 70 may include a lumen into which the distal end 1525 of the post 1520 may be coupled. The Y-adapter 70 may fluidly communicate the flow restriction device 1500 having the post 1520, with the catheter assembly 50, for example, via the extension tubing 60. Accordingly, the lumen 1522 of the post 1520 may define a linear fluid pathway with a reduced, small, or micro-sized diameter (as discussed below) through which fluid entering the flow restriction device 1500 from the catheter assembly 50, may flow through the flow restriction device 1500 for collection in the fluid collection device 40. For example, where blood is being withdrawn or collected from a patient, the medical fluid 15 may be blood, and the fluid collection device 40 may be a blood collection device. In some embodiments, the blood collection device 40 may be a luer lock access device (LLAD) or a syringe. Accordingly, during blood collection or withdrawal from the patient, the blood sample 15 may flow from the distal end 1525 of the post 1520, and into the LLAD 40 via the flowpath or micro-channel defined by the lumen 1522.

As described above, the post 1520 may be an elongate, thin tube with the lumen 1522 having a small, reduced, or micro-sized diameter. For example, in some embodiments, the lumen 1522 of the post 1520, that defines the flowpath or micro-channel along which fluid may flow from the distal end 1525 into the fluid collection device 40, may be formed by any of a length, a diameter, and a cross-sectional area as described above with reference to an optimized fluid pathway.

In order to draw fluid (e.g., blood) from the catheter assembly 50, the fluid collection device 40 may be inserted into and coupled to the inlet 1534 of the body portion 1510. In the coupled configuration of the body portion 1510 and the fluid collection device 40, the compressible valve member 1540 may be compressed distally by the fluid collection device 40 to fluidly communicate the proximal end 1523 of post 1520 and the fluid collection device 40. For example, as a male luer portion of the blood collection device 40 is inserted into the inlet 1534 of the body portion, the male luer portion of the blood collection device 40 may move or otherwise displace the head portion 1542 of the valve member 1540 and cause the valve member 1540 to compress in the distal direction. As the head portion 1542 is displaced distally, the proximal end 1523 of the post 1520 may be exposed to the exterior of the valve member 1540 via the slot 1550. The proximal end 1523 of the post 1520 may thus be fluidly coupled to the male luer portion of the blood collection device 40 via the slot 1550 of the compressible valve member 1540.

In operation, during blood collection or withdrawal from the patient, the blood 15 may flow from the patient's vein into the catheter assembly 50, through the extension tubing 60, and enter the distal end 1525 of the post 1540. Due to the presence of the post 1520 in the lumen 1543 of the base 1545, the blood 15 may be forced to flow into and through the flowpath or micro-channel defined by the lumen 1522, and exit the flow restriction device 1500 into the blood collection device 40 via the proximal end 1523 of the post 1520. Accordingly, during blood collection or withdrawal from the patient, the blood 15 may flow into the blood collection device 40 via the flowpath or micro-channel defined by the lumen 1522 having minimal diameter. The flow restriction device 1500 of the various embodiments described herein is advantageous over currently existing blood collection systems. For example, during blood draw with currently existing blood draw devices, blood cells may experience shear stress as they flow from the distal end to the proximal end of the blood collection systems. The maximum shear stress may be along the wall of the blood cell, often referred to as wall shear stress. Wall shear stress on blood cells is considered a major source of mechanical damage to blood cells causing hemolysis of the blood cells. In some embodiments, the continuous flowpath or micro-channel defined by the lumen 1522 having minimal diameter may facilitate increased flow resistance within the vascular access system to distribute the pressure differential and reduce shear stress experienced by the red blood cells of the blood 15. For example, the minimized diameter of the flowpath or micro-channel defined by the lumen 1522 may provide increased resistance to flow of the blood 15 and thereby decrease blood flow rate within the flow resistance device 1500. Since the decreased blood flow rate causes a reduction in shear stress experienced by the red blood cells of the blood 15, a risk of hemolysis during blood collection may advantageously be reduced.

FIG. 14C illustrates a cross-sectional view of a flow restriction device, in accordance with some embodiments of the present disclosure. FIG. 14D illustrates a cross-sectional view of a flow restriction device when coupled to a fluid collection device, in accordance with some embodiments of the present disclosure. As illustrated in FIGS. 14C and 14D, with continued reference to FIGS. 9A and 9B, in some embodiments, the flow restriction device 1503 may include a connector 1506 having a female luer portion 1532 with a lumen 1539 and disposed at a proximal end, and a male luer portion 1512 having a lumen 1519 and disposed at distal end. The female luer portion 1532 may be configured to couple to a fluid collection device 40, and the male luer portion 1512 may be configured to couple to a catheter assembly 50. In some embodiments, the flow restriction device 1503 may further include a compressible valve member 1540 mounted in the lumen 1539 of the female luer portion 1532 and fluidly coupled to the lumen 1519 of the male luer portion 1545. The compressible valve member 1540 may include an inner surface 1541 defining an internal chamber 1543 of the compressible valve member 1540. As depicted, the compressible valve member 1540 may have a head portion 1542 including a slot 1550, and a body portion 1544 extending distally from the head portion 1542. In some embodiments, the body portion 1544 may have an accordion shape, or any other similar compressible or collapsible shape. In some embodiments, the head portion 1542 having the slot 1550 may be a split-septum head portion.

According to various embodiments of the present disclosure, the flow restriction device 1503 may further include a post 1520 mounted in the female luer portion 1532 and extending into the internal chamber 1543 of the compressible valve member. Accordingly, the compressible valve member 1540 may be mounted surrounding the post 1520. The post 1520 may be fluidly communicated with the lumen 1519 of the male luer portion 1512, which may in turn be fluidly communicated with a catheter assembly 50. In some embodiments, the post 1520 may be press-fit into the lumen 1539 of the female luer portion 1532. However, the various embodiments of the present disclosure are not limited to the aforementioned configuration. In some embodiments, the post 1520 may be fastened, attached, or otherwise coupled in the lumen 1539 of the female luer portion 1532 by any other suitable joining means. In some embodiments, the female luer portion 1532 may further include a support portion 1546 at a distal end thereof. The post 1520 may be mounted on the support portion 1546 for fluidly communicating the lumen 1519 of the male luer portion 1512 with the fluid collection device 40.

In some embodiments, the post 1520 may be in the form of an elongate tube having a proximal end 1523, a distal end 1525, and the lumen 1522 extending therethrough. In some embodiments, the post 1520 may have a shape that tapers from the distal end 1525 to the proximal end 1523 of the post 1520. Accordingly, in some embodiments, a shape or profile of the lumen 1522 may also taper from the distal end 1525 to the proximal end 1523 of the post 1520. As depicted, the post 1520 may extend from the distal end 1523 into the internal chamber 1541 of the compressible valve member 1540 which is disposed in the lumen 1539 of the female luer portion 1532. The lumen 1522 of post 1520 may define a flowpath or micro-channel along which a fluid may flow from the distal end 1525 into the fluid collection device 40. The post 1520 may fluidly couple the blood collection device 40 with the catheter assembly 50 via the lumen 1519 of the male luer portion 1512. For example, with continued reference to FIGS. 9A and 9B, in some embodiments, a leg 72 of the Y-adapter 70 may be coupled to the flow restriction device 1503. The leg 72 of Y-adapter 70 may include a lumen into which the distal end 1516 of the male luer portion 1512 may be coupled. The Y-adapter 70 may fluidly communicate the flow restriction device 1503 having the post 1520, with the catheter assembly 50, for example, via the extension tubing 60. Accordingly, the lumen 1522 of the post 1520 may define a linear fluid pathway with a reduced, small, or micro-sized diameter (as discussed below) through which fluid entering the flow restriction device 1503 from the catheter assembly 50, may flow through the flow restriction device 15000 for collection in the fluid collection device 40. For example, where blood is being withdrawn or collected from a patient, the medical fluid 15 may be blood, and the fluid collection device 40 may be a blood collection device. In some embodiments, the blood collection device 40 may be a luer lock access device (LLAD). Accordingly, during blood collection or withdrawal from the patient, the blood sample 15 may flow from the distal end 1525 of the post 1520, and into the LLAD 40 via the flowpath or micro-channel defined by the lumen 1522.

As described above, the post 1520 may be an elongate, thin tube with the lumen 1522 having a small, reduced or micro-sized diameter. For example, in some embodiments, the lumen 1522 of the post 1520, that defines the flowpath or micro-channel along which fluid may flow from the distal end 1525 into the fluid collection device 40, may be formed by any of a length, a diameter, and a cross-sectional area as described above with reference to an optimized fluid pathway.

In order to draw fluid (e.g., blood) from the catheter assembly 50, the fluid collection device 40 may be inserted into and coupled to the inlet 1535 of the female luer portion 1532. In the coupled configuration of the female luer portion 1532 and the fluid collection device 40, the compressible valve member 1540 may be compressed distally by the fluid collection device 40 to fluidly communicate the proximal end 1523 of post 1520 and the fluid collection device 40. For example, as a male luer portion 1502 of the blood collection device 40 is inserted into the inlet 1535 of the female luer portion 1532, the male luer portion 1502 of the blood collection device 40 may move or otherwise displace the head portion 1542 of the valve member 1540 and cause the valve member 1540 to compress in the distal direction. As the head portion 1542 is displaced distally, the proximal end 1523 of the post 1520 may be exposed to the inlet of the male luer portion 1502 of the blood collection device 40 via the slot 1550. The proximal end 1523 of the post 1520 may thus be fluidly coupled to the male luer portion of the blood collection device 40 via the slot 1550 of the compressible valve member 1540.

In operation, during blood collection or withdrawal from the patient, the blood 15 may flow from the patient's vein into the catheter assembly 50, through the extension tubing 60, and enter the lumen 1519 at the distal end 1516 of the male luer portion 1512 of flow restriction device 1503. The blood 15 may then flow proximally towards the female luer portion 1532. Due to the presence of the post 1520 in the lumen 1543 of the base 1545, the blood 15 may be forced to flow into the distal end 1525 of the post 1540 and through the flowpath or micro-channel defined by the lumen 1522, and exit the flow restriction device 1503 into the blood collection device 40 via the proximal end 1523 of the post 1520. Accordingly, during blood collection or withdrawal from the patient, the blood 15 may flow into the blood collection device 40 via the flowpath or micro-channel defined by the lumen 1522 having minimal diameter. The flow restriction device 1503 of the various embodiments described herein is advantageous over currently existing blood collection systems. For example, during blood draw with currently existing blood draw devices, blood cells may experience shear stress as they flow from the distal end to the proximal end of the blood collection systems. The maximum shear stress may be along the wall of the blood cell, often referred to as wall shear stress. Wall shear stress on blood cells is considered a major source of mechanical damage to blood cells causing hemolysis of the blood cells. In some embodiments, the continuous flowpath or micro-channel defined by the lumen 1522 having minimal diameter may facilitate increased flow resistance within the vascular access system to distribute the pressure differential and reduce shear stress experienced by the red blood cells of the blood 15. For example, the minimized diameter of the flowpath or micro-channel defined by the lumen 1522 may provide increased resistance to flow of the blood 15 and thereby decrease blood flow rate within the flow resistance device 1503. Since the decreased blood flow rate causes a reduction in shear stress experienced by the red blood cells of the blood 15, a risk of hemolysis during blood collection may advantageously be reduced.

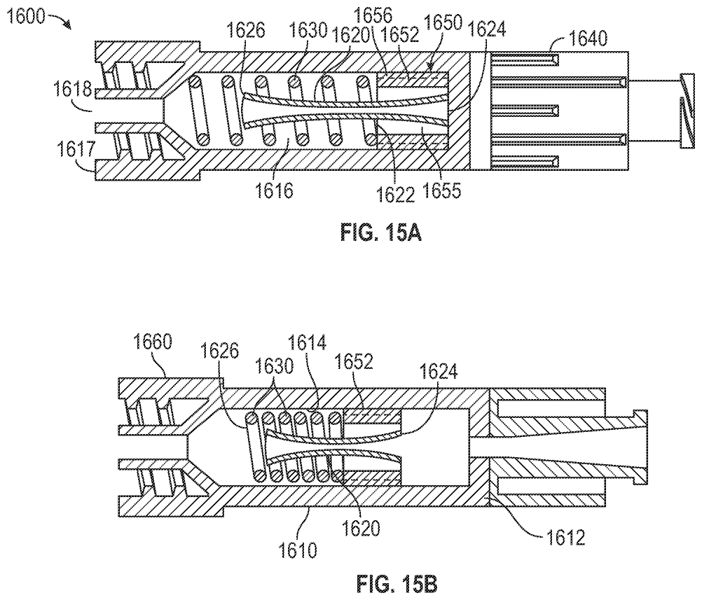

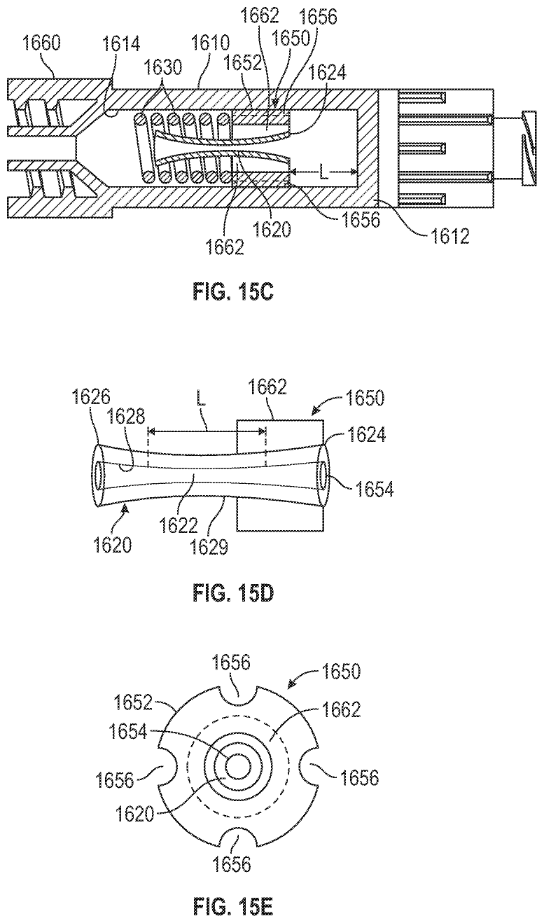

FIG. 15A illustrates a cross-sectional view of a flow restriction device 1600 in a fluid draw position, in accordance with some embodiments of the present disclosure. FIG. 15B illustrates a cross-sectional view of the flow restriction device 1600 of FIG. 15A in a fluid infusion position, in accordance with some embodiments of the present disclosure. FIG. 15C illustrates a perspective view of a flow-restricting post or a tubing 1620 of the flow restriction device of FIG. 15A, in accordance with some embodiments of the present disclosure. FIG. 15D illustrates a cross-sectional view of a slider 1650 of the flow restriction device of FIG. 15A, in accordance with some embodiments of the present disclosure. As illustrated in FIGS. 15A-15E, with continued reference to FIGS. 9A and 9B, in some embodiments, the flow restriction device 1600 may include a housing 1610 having a proximal end 1612, an inner surface 1614 defining an internal chamber 1616 of the housing 1610. In some embodiments the internal chamber 1616 of the housing 1610 may be fluidly coupled to a reciprocal flow connector 1640, for example, but not limited to a needleless connector. The flow restriction device 1600 may further include a male luer portion 1660 defining a distal end 1617 of the housing 1610. The male luer portion 1660 may have a lumen fluidly communicated with the internal chamber 1616. In some embodiments, the slider 1650 is reciprocally mounted in the internal chamber 1616. As depicted, the slider 1650 may have a proximal end 1624, a proximal face 1662, a distal end 1622, a mounting aperture 1655 extending from the proximal end 1624 to the distal end 1622 of the slider 1650, and a plurality of flow apertures 1656 extending from the proximal end 1624 to the distal end 1622 of the slider 1650 and surrounding the mounting aperture 1655. In some embodiments, the plurality of flow apertures 1656 may be at least four flow apertures 1656. However, the various embodiments of the present disclosure are not limited to the aforementioned configuration. In some embodiments, there may be less than four flow apertures 1656, but greater than one flow aperture 1656. In some embodiments, the plurality of flow apertures 1656 may extend longitudinally about an outer periphery 1652 of the slider 1650. For example, in some embodiments the plurality of flow apertures 1656 may extend longitudinally about the outer periphery 1652 of the slider 1650. In some embodiments, the slider 1650 may be formed of a polyisopropene seal material.

As depicted in FIGS. 15A and 15B, the flow restriction device 1600 may further include the flow-restricting post in the form of tubing 1620 having an inner surface 1628 defining a lumen 1625 extending therethrough and between a proximal end 1627 and a distal end 1626 of the tubing 1620. The tubing 1620 may be mounted in the mounting aperture 1655 and may extend from the proximal end 1624 of the slider 1650 distally through the distal end 1622 of the slider assembly 1650. In accordance with some embodiments of the present disclosure, the lumen 1625 may define a flowpath or micro-channel along which a fluid (e.g., blood) may flow from the internal chamber 1616 into the fluid collection device 40, for example via the connector 1640.

In some embodiments, a leg 72 of the Y-adapter 70 may be coupled to the flow restriction device 1600. For example, the leg 72 of Y-adapter 70 may include a lumen into which the distal end 1617 of the housing 1610 having the tubing 1620 mounted therein may be coupled. The Y-adapter 70 may fluidly communicate the flow restriction device 110, and tubing 1620 mounted therein, with the catheter assembly 50, for example, via the extension tubing 60. Accordingly, the lumen 1625 of the tubing 1620 may define a linear fluid pathway with a reduced, small, or micro-sized diameter (as discussed below) through which fluid entering the flow restriction device 1600 from the catheter assembly, may flow through the flow restriction device 1600 for collection in the fluid collection device 40. For example, where blood is being withdrawn or collected from a patient, the medical fluid 15 may be blood, and the fluid collection device 40 may be a blood collection device. In some embodiments, the blood collection device may be a luer lock access device (LLAD). Accordingly, during blood collection or withdrawal from the patients, the blood sample 15 may flow from the distal end 1617 of the housing 1610 into the LLAD 40 via the flowpath or micro-channel defined by lumen 1625.

In some embodiments, at least a portion of the tubing 1620 may be in the form of a longitudinal body uniformly extending in the internal chamber 1616 of the housing 1610. In some embodiments, at least one of a diameter of the tubing at the proximal end 1627 thereof and a diameter of the tubing at the distal end 1626 thereof is greater than a diameter of the portion L of the tubing 1620 which comprises a longitudinal body uniformly extending in the internal chamber 1616 of the housing 1610. For example, in some embodiments the tubing 1620 is flared radially outward from the portion L of the tubing 1620 that comprises a longitudinal body uniformly extending in the internal chamber 1616 of the housing 1610 to at least one of the proximal end 1627 and the distal ends 1626 of the tubing 1620.

In some embodiments, the portion L of the tubing 1620 which comprises a longitudinal body uniformly extending in the internal chamber 1616 of the housing 1610 may be an elongate, thin tube with lumen 1625 having a small, reduced or micro-sized diameter. For example, in some embodiments, the lumen 1625 of the portion L of the tubing 1620 that defines the flowpath or micro-channel along which fluid may flow from the distal end 1617 into the fluid collection device 40, for example via the connector 1640, may be formed by any of a length, a diameter, and a cross-sectional area as described above with reference to an optimized fluid pathway.

According to various embodiments of the present disclosure, the flow restriction device 1600 may further include a spring member 1630 mounted in the internal chamber 1616 surrounding at least a portion of the tubing 1620. Accordingly, the tubing 1620 may be spring-loaded by the spring member 1630. In operation, during blood draw or very light infusion, when the slider 1650 is under draw or very light infusion pressures, the spring member 1630 in its extended/uncompressed state exerts a force to bias the proximal end 1624 of the slider 1650 proximally against the inner surface 1614 of the housing 1610. Accordingly, the proximal end 1624 of the slider 1650 as well as the plurality of flow apertures 1656 would remain sealed against the inner surface 1614 of the housing thereby occluding fluid flow into the connector 1640 via the plurality of flow apertures 1656. Since the fluid paths between the plurality of flow apertures 1656 and the connector 1640 is occluded, during blood draw, blood entering the lumen 1618 of the male luer portion 1660 of flow restriction device 1600 may flow into the connector 1640 only through the lumen 1625 of the tubing 1620.

When the fluid 15 is blood being withdrawn from a patient, blood cells may experience shear stress as they flow from the catheter assembly 50 into the blood collection device 40. For example, the maximum shear stress may be along the wall of the blood cell, often referred to as wall shear stress. Wall shear stress on blood cells is considered a major source of mechanical damage to blood cells causing hemolysis of the blood cells. In some embodiments, the lumen 1625 of the tubing 1620 having a reduced or micro-sized diameter may facilitate increased flow resistance within the vascular access system to distribute the pressure differential and reduce shear stress experienced by the red blood cells of the blood 15. The minimized diameter of the first fluid pathway or micro-channel defined by the lumen 1625 of the tubing 1620 may provide increased resistance to flow of the blood 15 and thereby decrease blood flow rate within the flow resistance device 1600. Since the decreased blood flow rate causes a reduction in shear stress experienced by the red blood cells of the blood 15, a risk of hemolysis during blood collection may advantageously be reduced.

When the slider 1650 is subject to a distally-direction fluid pressure, for example infusion pressures, the infusion fluid, e.g., intravenous (IV) fluid, the IV fluid would initially flow from the connector 1640 into the internal chamber 1616 of the housing via the lumen 1625 of the tubing 1620. As the infusion pressure increases, the slider 1650 may be configured to move distally away from the inner surface 1614 of the housing 1610 and compress the spring member 1630. As depicted, each of the plurality of apertures 1656 and the inner surface 1614 of the internal chamber 1616 may define a flowpath through which the fluid, e.g., intravenous (IV) fluid flows from the connector into the lumen of the male luer portion 1660 when the slider 1650 is subject to the distally-directioned fluid pressure. Due to the separation of the proximal end 1624 of the slider from the inner surface 1614 of the housing 1610, the flowpaths through the plurality of flow apertures 1656 are opened. Accordingly, fluid, e.g., infusion fluid, may flow from the connector 1640 into the lumen 1618 of the male luer portion 1660 via both the lumen 1625 of the tubing 1620 and the plurality of secondary flow apertures 1656.

Accordingly, the spring member 1630 keeps the slider 1650 with tubing 1620 mounted therein biased and sealed up against the inner surface of the housing 1610, so that in slight vacuum (i.e., suction) and very light infusion pressures, the fluid would flow through the tubing 1620 having the lumen 1625 defining the micro-channel. The initial burst of a flush would also flow through the tubing 1620 having the lumen 1625 defining the micro-channel, thereby enabling some higher-pressure flushing. When infusion pressure gets greater, then the spring member 1630 may be compressed by the distal movement D of the slider 1650, and the plurality of flow apertures 1656 are opened up around the outer diameter of the slider, such that multiple flow paths, i.e., through the lumen 1625 of the tubing 1620 and the plurality of flow apertures 1656, are open for flushing or infusion. Thus, flushing of all fluid paths is fully enabled.

FIG. 16A illustrates a blood collection system 1700, in accordance with some embodiments of the present disclosure. FIG. 16B illustrates a blood collection system 1700, in accordance with some embodiments of the present disclosure. According to various embodiments of the present disclosure, a blood collection system 1700 may include a blood collection device 1702 including a container 1710 having an outer surface 1714, an inner surface 1722 defining an internal chamber 1750, a needle 1730 extending proximally from the inner surface 1722, and a connection portion 1720 extending distally from the outer surface 1714. In some embodiments, the blood collection device 1702 may be a luer lock access device (LLAD). In some embodiments, the blood collection device 1702 may include a VACUTAINER® blood collection tube, available from Becton Dickinson & Company.

In some embodiments, the blood collection system may further include a flow restriction device 1732 fluidly coupled to the connection portion 1720 of the blood collection device 1702. In some embodiments, the flow restriction device 1732 may be pre-attached or integrally formed with the blood collection device 1702. As depicted, the flow restriction device 1732 may be a cannula 1740 having a lumen 1742 defining an internal flowpath therethrough. The internal flowpath may be fluidly coupled to a lumen 1734 of the needle 1730 for delivering blood drawn from a patient to the needle 1730.

In some embodiments, the cannula 1740 may be an elongate, thin tube with the lumen 1742 having a small, reduced, or micro-sized diameter. For example, in some embodiments, the lumen 1742 of the cannula 1740, that may define a flowpath or micro-channel along which fluid may flow into the needle 1730, may be formed by any of a length, a diameter, and a cross-sectional area as described above with reference to an optimized fluid pathway. Accordingly, during blood collection or withdrawal from the patient, for example using a VACUTAINER® blood collection tube, the blood may be drawn into the needle 1730 under vacuum via the flowpath or micro-channel defined by the lumen 1742 having minimal diameter.

FIG. 16B illustrates a blood collection system 1800, in accordance with some embodiments of the present disclosure. According to various embodiments of the present disclosure, a blood collection system 1800 may include a blood collection device 1802 including a container 1710 having an outer surface 1714, an inner surface 1722 defining an internal chamber 1750, and a connection portion 1720 extending distally from the outer surface 1714 and having a lumen 1724 extending therethrough. In some embodiments, the blood collection device 1802 may be a luer lock access device (LLAD). In some embodiments, the blood collection device 1802 may include a VACUTAINER® blood collection tube, available from Becton Dickinson & Company.

In some embodiments, the blood collection system may further include a flow restriction device 1832 extending proximally from the inner surface 1722 and fluidly coupled to the lumen 1724 of the connection portion 1720 of the blood collection device 1702. In some embodiments, the flow restriction device 1832 may be pre-attached or integrally formed with the blood collection device 1802. As depicted, the flow restriction device 1732 may be a cannula 1740 having a lumen 1742 defining an internal flowpath therethrough. The internal flowpath may be fluidly coupled to a catheter assembly, via the lumen 1724 of the connection portion, for delivering blood drawn from a patient into the blood collection device 1802.

In some embodiments, similar to the blood collection system 1700, the cannula 1740 may be an elongate, thin tube with the lumen 1742 having a small, reduced or micro-sized diameter. For example, in some embodiments, the lumen 1742 of the cannula 1740, that may define a flowpath or micro-channel along which fluid may flow into the container 1710, may by formed by any of a length, a diameter, and a cross-sectional area as described above with reference to an optimized fluid pathway. Accordingly, during blood collection or withdrawal from the patient, for example using a VACUTAINER® blood collection tube, the blood may be drawn into the flowpath or micro-channel defined by the lumen 1742 having minimal diameter under vacuum.

The blood collection systems 1700 and 1800 of the various embodiments described herein having a flow restriction cannula 1740 integrated into the blood collection device 1702, 1802 is advantageous over currently existing blood collection systems. For example, during blood draw with currently existing blood draw devices, blood cells may experience wall shear stress as they flow from the distal end to the proximal end of the blood collection systems. Wall shear stress on blood cells is considered a major source of mechanical damage to blood cells causing hemolysis of the blood cells. The flowpath or micro-channel defined by the lumen 1742 having minimal diameter may facilitate increased flow resistance within the vascular access system to distribute the pressure differential and reduce shear stress experienced by the red blood cells of the blood. For example, the minimized diameter of the flowpath or micro-channel defined by the lumen 1742 may provide increased resistance to flow of the blood and thereby decrease blood flow rate within the flow cannula 1740. Since the decreased blood flow rate causes a reduction in shear stress experienced by the red blood cells of the blood, a risk of hemolysis during blood collection may advantageously be reduced.

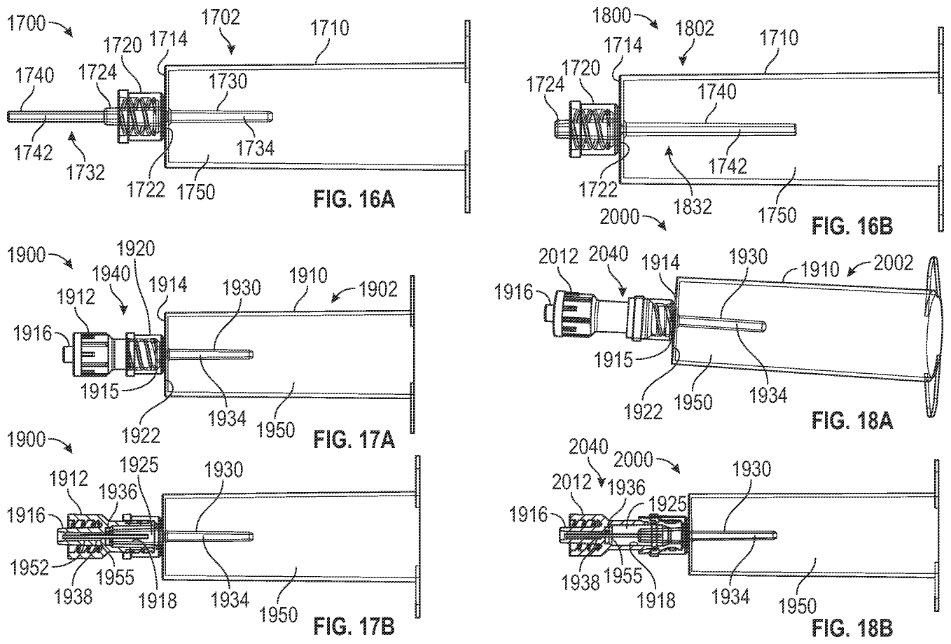

FIG. 17A illustrates a blood collection system 1900, in accordance with some embodiments of the present disclosure. FIG. 17B illustrates a cross-sectional view of the blood collection system 1900 of FIG. 17A, in accordance with some embodiments of the present disclosure. According to various embodiments of the present disclosure, a blood collection system 1900 may include a blood collection device 1902 including a container 1910 having an outer surface 1914, an inner surface 1922 defining an internal chamber 1950, a needle 1930 extending proximally from the inner surface 1922, and a connection portion 1920 extending distally from the outer surface 1914. In some embodiments, the blood collection device 1902 may be a luer lock access device (LLAD). In some embodiments, the blood collection device 1902 may include a VACUTAINER® blood collection tube, available from Becton Dickinson & Company.

In some embodiments, the blood collection system may further include a flow restriction device 1940 engaged with the connection portion 1920 and fluidly coupled to a lumen 1934 of the needle 1930. In some embodiments, the flow restriction device 1940 may be pre-attached or integrally formed with the blood collection device 1902. As depicted in FIGS. 17A and 17B, the flow restriction device 1940 may include a connector 1912 having a proximal end 1915 disposed in the connection portion 1920, a distal end 1916 configured to couple to a catheter assembly, and an internal surface 1918 defining an inner lumen 1925. The flow restriction device 1940 may further include a cannula 1955 mounted in the inner lumen 1918 extending from the distal end 1916 of the connector 1912 into the connection portion 1920. The lumen 1952 of the cannula 1955 may define a flowpath along which blood flows from the distal end 1916 into the lumen 1934 of the needle 1930.

In some embodiments, the cannula 1955 may be an elongate, thin tube with the lumen 1952 having a small, reduced or micro-sized diameter. For example, in some embodiments, the lumen 1952 of the cannula 1955, that may define a flowpath or micro-channel along which fluid may flow into the needle 1930, may be formed by any of a length, a diameter, and a cross-sectional area as described above with reference to an optimized fluid pathway. Accordingly, during blood collection or withdrawal from the patient, for example using a VACUTAINER® blood collection tube, the blood may be drawn into the needle 1930 under vacuum via the flowpath or micro-channel defined by the lumen 1952 having minimal diameter.

In some embodiments, the connector 1912 may further include a support portion 1936 disposed between the proximal and distal ends 1915 and 1916. The support portion 1936 may have a proximal end, a distal end, and a central mounting aperture 1938 extending from the proximal to the distal end. As depicted, the cannula 1955 may be mounted in the central mounting aperture 1938 for fluidly communicating the flow restriction device with the catheter assembly.

FIG. 18A illustrates a blood collection system 2000, in accordance with some embodiments of the present disclosure. FIG. 18B illustrates a cross-sectional view of the blood collection system 2000 of FIG. 18A, in accordance with some embodiments of the present disclosure. According to various embodiments of the present disclosure, a blood collection system 2000 may include a blood collection device 2002 including a container 1910 having an outer surface 1914, an inner surface 1922 defining an internal chamber 1950, a needle 1930 extending proximally from the inner surface 1922, and a connection portion 1920 extending distally from the outer surface 1914. In some embodiments, the blood collection device 2002 may be a luer lock access device (LLAD). In some embodiments, the blood collection device 2002 may include a VACUTAINER® blood collection tube, available from Becton Dickinson & Company.

In some embodiments, the blood collection system may further include a flow restriction device 2040 engaged with the connection portion 1920 and fluidly coupled to a lumen 1934 of the needle 1930. In some embodiments, the flow restriction device 1940 may be pre-attached or integrally formed with the blood collection device 1902. The flow restriction device 2040 may include a connector 2012 having a proximal end 1915 disposed in the connection portion 1920, a distal end 1916 configured to couple to a catheter assembly, and an internal surface 1918 defining an inner lumen 1925. The flow restriction device 2040 may have features similar to the features of the flow restriction device 1940 described above, a detailed description of which shall be omitted with respect to FIGS. 18A and 18B. However, the flow restriction device 2040 may differ from the flow restriction device 1940 in that the cannula 1955 of flow restriction device 2040 may be mounted in the inner lumen 1925 of the connector extending from the distal end 1915 to the proximal end of the connector 2012. In particular, the cannula 1955 of the flow restriction device 2040 may be concealed within the body of the connector 2012, in contrast to the cannula 1955 of the flow restriction device 1940 which extends into the blood collection device 1902.

The blood collection systems 1900 and 2000 of the various embodiments described herein having a flow restriction cannula 1955 integrated into the blood collection device 1902, 2002 is advantageous over currently existing blood collection systems. For example, during blood draw with currently existing blood draw devices, blood cells may experience wall shear stress as they flow from the distal end to the proximal end of the blood collection systems. Wall shear stress on blood cells is considered a major source of mechanical damage to blood cells causing hemolysis of the blood cells. The flowpath or micro-channel defined by the lumen 1952 having minimal diameter may facilitate increased flow resistance within the vascular access system to distribute the pressure differential and reduce shear stress experienced by the red blood cells of the blood. For example, the minimized diameter of the flowpath or micro-channel defined by the lumen 1952 may provide increased resistance to flow of the blood and thereby decrease blood flow rate within the flow cannula 1955. Since the decreased blood flow rate causes a reduction in shear stress experienced by the red blood cells of the blood, a risk of hemolysis during blood collection may advantageously be reduced.

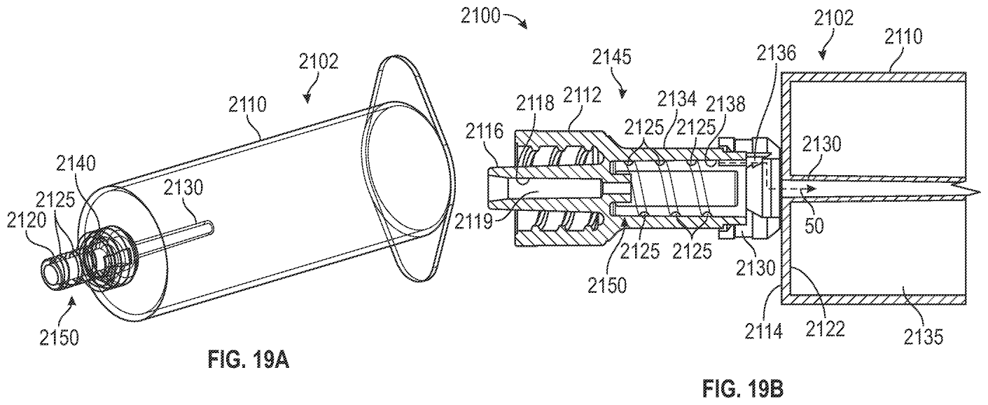

FIG. 19A illustrates a perspective view of a blood collection system 2100, in accordance with some embodiments of the present disclosure. FIG. 19B illustrates a cross-sectional view of the blood collection system 2100 of FIG. 19A, in accordance with some embodiments of the present disclosure. According to various embodiments of the present disclosure, a blood collection system 2100 may include a blood collection device 2102 including a container 2110 having an outer surface 2114, an inner surface 2122 defining an internal chamber 2135, a needle 2130 extending proximally from the inner surface 2122, and a connection portion 2140 extending distally from the outer surface 2114. In some embodiments, the blood collection device 2102 may be a luer lock access device (LLAD). In some embodiments, the blood collection device 2102 may include a VACUTAINER® blood collection tube, available from Becton Dickinson & Company.

In some embodiments, the connection portion 2140 may include an insert portion 2150 having an outer surface 2120 having a continuous non-linear channel 2125 recessed therein. The blood collection system may further include a connector 2112 configured to fluidly couple the blood collection device 2102 to a catheter assembly. The connector 2112 may include a first connection portion 2134 at a proximal end thereof and a second connection portion 2116 at a distal end thereof. As depicted in FIG. 19B, the first connection portion 2134 may have an inner surface 2138 defining a lumen into which the insert portion 2150 is coupled.

In the coupled configuration of the of the connector 2112 and the insert portion 2150, the inner surface 2138 of the first connection portion 2134 may encase, surround, or otherwise envelope the outer surface of the insert portion 2150 such that the continuous non-linear channel 2125 and the inner surface 2138 of the first connection portion 2134 define a non-linear fluid pathway along which a fluid (e.g., blood) may flow from a lumen 2119 of the second connection portion 2116 of the connector 2112 into the fluid collection device 2102.

In some embodiments, the continuous non-linear channel 2125 may form a coil shape, an S-shape, or another suitable non-linear, winding shape. For example, the continuous non-linear channel 2125 may have a coil shape (which may include a spiral) recessed in the outer surface 2120 of the insert portion 2150. In some embodiments, the continuous non-linear channel 2125 may have an S-shape recessed in the outer surface of the insert portion 2150. The aforementioned configuration is advantageous in that the spiral, coil shape, S-shape, or other suitable non-linear, winding shape of the continuous non-linear channel 2125—by virtue of its wrapping around the outer surface of the insert portion 2150—may increase a length of the fluid pathway defined by the continuous non-linear channel 2125 through which the blood sample flows as compared with a linear fluid pathway.

In some embodiments, the continuous non-linear channel 2125 may have a small, reduced or micro-sized diameter. For example, in some embodiments, the continuous non-linear channel 2125 that defines the flowpath or micro-channel along which fluid may flow from the connector 2112 into the fluid collection device 2102 may be formed by any of a length, a diameter, and a cross-sectional area as described above with reference to an optimized fluid pathway.

As previously described, when the blood is being withdrawn from a patient, blood cells may experience wall shear stress as they flow from the catheter assembly into the blood collection device. For example, the maximum shear stress may be along the wall of the blood cell, often referred to as wall shear stress. The continuous non-linear channel 2125 having a reduced or micro-sized diameter may facilitate increased flow resistance within the vascular access system to distribute the pressure differential and reduce shear stress experienced by the red blood cells of the blood. The minimized diameter of the continuous non-linear channel 2125 may provide increased resistance to flow of the blood and thereby decrease blood flow rate within the flow blood collection device 2102. Since the decreased blood flow rate causes a reduction in shear stress experienced by the red blood cells of the blood, a risk of hemolysis during blood collection may advantageously be reduced.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A flow restriction device, comprising: a first connector comprising a proximal end, a distal end, and an internal surface defining an inner lumen, the first connector configured to couple to a catheter assembly; a second connector coupled to the proximal end of the first connector and configured to couple to a fluid collection device; a cannula mounted in the inner lumen extending from the distal end of the first connector into the second connector, wherein a lumen of the cannula defines a first flowpath along which a fluid flows from the distal end into the fluid collection device, and an annulus is defined between an outer surface of the cannula and the internal surface of the first connector, the annulus defining a second flowpath along which a fluid flows from the proximal end to the distal end and into the catheter assembly, and a check valve mounted in the annulus at the proximal end of the first connector, and sleeved over at least a portion of the cannula, the check valve configured to (i) prevent fluid flowing from the distal end into the fluid collection device via the second flowpath, and (ii) allow fluid to flow from the second connector into the first connector and the catheter assembly via the second flowpath.

Clause 2. The flow restriction device of clause 1, wherein a cross-sectional area of the annulus about a plane transverse to a central longitudinal axis of the first connector is greater than a cross-sectional area of the lumen about the plane.

Clause 3. The flow restriction device of clause 2, wherein the second connector comprises a proximal end, a distal end, and a support portion disposed between the proximal and distal ends, and wherein the support portion has a proximal end, a distal end, and comprises a central mounting aperture extending from the proximal to the distal end, a proximal end of the cannula being mounted in the central mounting aperture for fluidly communicating the flow restriction device with the fluid collection device.

Clause 4. The flow restriction device of clause 3, wherein the support portion further comprises plurality of fluid channels disposed radially outward of and surrounding the central mounting aperture.

Clause 5. The flow restriction device of clause 4, wherein the plurality of fluid channels extend from the proximal to the distal end of the support portion to fluidly communicate the second connector with the annulus, the plurality of fluid channels defining at least a portion of the second flowpath.

Clause 6. The flow restriction device of any of clauses 3 to 5, wherein the cannula comprises a notch at position corresponding to a distal end of the check valve, the notch fluidly connecting the lumen of the cannula with the annulus.

Clause 7. The flow restriction device of any of clauses 2 to 6, wherein the first connector further comprises a support portion disposed between the proximal and distal ends of the first connector, and wherein the support portion of the first connector has a proximal end, a distal end, and comprises a central mounting aperture extending from the proximal to the distal end, the cannula mounted in the central mounting aperture of the support portion of the first connector for fluidly communicating the flow restriction device with the catheter assembly.

Clause 8. The flow restriction device of clause 7, wherein the support portion of the first connector further comprises a plurality of fluid channels disposed radially outward of and surrounding the central mounting aperture of the first connector.

Clause 9. The flow restriction device of clause 8, wherein the plurality of fluid channels extend from the proximal to the distal end of the support portion of the first connector to fluidly communicate the annulus with the catheter assembly, the plurality of fluid channels defining at least a portion of the second flowpath.

Clause 10. The flow restriction device of clause 9, wherein the fluid flowing from the distal end into the fluid collection device via the first flowpath comprises blood and the fluid collection device comprises a blood collection device.

Clause 11. The flow restriction device of clause 10, wherein the fluid flowing from the second connector into the first connector and the catheter assembly via the second flowpath comprises an intravenous (IV) fluid.

Clause 12. A flow restriction device, comprising: a first connector comprising a proximal end, a distal end, and an internal surface defining an inner lumen, the distal end configured to couple to a catheter assembly; a second connector comprising a proximal end, a distal end, and an internal surface defining a lumen of the second connector, the second connector coupled to the proximal end of the first connector and configured to couple to a fluid collection device; a cannula mounted in the lumen of the second connector and extending distally into the inner lumen of the first connector, wherein: a lumen of the cannula defines a first flowpath along which a fluid flows from the distal end of the first connector into the fluid collection device; and an annulus is defined between an outer surface of the cannula and the internal surface of the first connector, the annulus defining a second flowpath along which a fluid flows from the proximal end to the distal end and into the catheter assembly; and a check valve mounted in the annulus at the proximal end of the first connector, and sleeved over at least a portion of the cannula, the check valve configured to (i) prevent fluid flowing from the distal end of the first connector into the fluid collection device via the second flowpath, and (ii) allow fluid to flow from the second connector into the first connector and the catheter assembly via the second flowpath.

Clause 13. The flow restriction device of clause 12, wherein a cross-sectional area of the annulus about a plane transverse to a central longitudinal axis of the first connector is greater than a cross-sectional area of the lumen about the plane.

Clause 14. The flow restriction device of clause 13, wherein the second connector further comprises a support portion disposed between the proximal and distal ends, and wherein the support portion has a proximal end, a distal end, and comprises a central mounting aperture extending from the proximal to the distal end, a proximal end of the cannula being mounted in the central mounting aperture for fluidly communicating the cannula with the fluid collection device.

Clause 15. The flow restriction device of clause 14, wherein the support portion further comprises plurality of fluid channels disposed radially outward of and surrounding the central mounting aperture.

Clause 16. The flow restriction device of clause 15, wherein the plurality of fluid channels extend from the proximal to the distal end of the support portion to fluidly communicate the second connector with the annulus, the plurality of fluid channels defining at least a portion of the second flowpath.

Clause 17. The flow restriction device of any of clauses 14 to 16, wherein the cannula comprises a notch at position corresponding to a distal end of the check valve, the notch fluidly connecting the lumen of the cannula with the annulus.

Clause 18. A flow restriction device, comprising: a distal connector configured to couple to a catheter assembly, the distal connector comprising a first connection portion at a proximal end thereof and a second connection portion at a distal end thereof, the first connection portion comprising an inner surface defining a lumen; a proximal connector coupled to the distal connector and configured to couple to a fluid collection device, the proximal connector comprising an insert portion for inserting into the lumen of the first connection portion, the insert portion including an outer surface having a continuous non-linear channel recessed therein, wherein the inner surface of the first connection portion encases the outer surface of the insert portion such that the continuous non-linear channel and the inner surface of the first connection portion define a non-linear fluid pathway along which a fluid flows from the distal connector into the fluid collection device.

Clause 19. The flow restriction device of clause 18, wherein the continuous non-linear channel comprises a continuous groove having a coil shape recessed in the outer surface.

Clause 20. The flow restriction device of any of clauses 18 and 19, wherein the continuous non-linear channel comprises a continuous groove having an S-shape recessed in the outer surface.

Clause 21. The flow restriction device of any of clauses 18 to 20, wherein the fluid flowing from the distal end into the fluid collection device comprises blood and the fluid collection device comprises a blood collection device.

Clause 22. The flow restriction device of clause 21, wherein the blood collection device comprises a luer lock access device.

Clause 23. The flow restriction device of any of clauses 18 to 22, wherein the insert portion further comprises an inner surface defining a lumen of the insert portion, the lumen forming an internal flowpath along which a fluid flows from the proximal connector into the catheter assembly via the distal connector.

Clause 24. The flow restriction device of clause 23, further comprising a check valve disposed in the lumen of the insert portion, the check valve configured to (i) prevent fluid flowing from the distal connector into the proximal connector via the lumen of the insert portion, and (ii) allow fluid to flow from the proximal connector into the distal connector via the lumen of the insert portion.

Clause 25. The flow restriction device of clause 24, wherein the fluid flowing from the proximal connector into the catheter assembly via the distal connector comprises an intravenous (IV) fluid.

Clause 26. The flow restriction device of any of clauses 23 to 25, wherein the internal flowpath in which the fluid flows from the proximal connector into the catheter assembly via the distal connector comprises a linear flowpath.

Clause 27. The flow restriction device of any of clauses 23 to 26, wherein the continuous non-linear channel comprises a first diameter, the internal flowpath comprises a second diameter, and the first diameter is smaller than the second diameter.

Clause 28. A flow restriction device, comprising: a distal connector portion configured to couple to a catheter assembly, the distal connector portion comprising an internal surface defining a lumen thereof; a proximal connector portion extending proximally from the distal connector portion, and configured to couple to a fluid collection device, the proximal connector portion comprising an internal surface defining a lumen fluidly connected to the lumen of the distal connector portion; a plug disposed in the lumen of the proximal connector portion, the plug comprising a head portion and a body portion extending proximally from the head portion, and the body portion comprising a plurality of threads extending along an outer surface of the body portion, wherein the internal surface of the proximal connector portion encases the outer surface of the body portion to define a continuous non-linear channel along which a fluid flows from the distal connector portion into the fluid collection device via the proximal connector portion when the fluid collection device is coupled to the proximal connector portion.

Clause 29. The flow restriction device of clause 28, wherein the continuous non-linear channel is defined along spacing between adjacent threads of the plurality of threads.

Clause 30. The flow restriction device of clause 29, wherein the continuous non-linear channel comprises a continuous groove having a coil shape recessed in the outer surface.

Clause 31. The flow restriction device of any of clauses 29 to 30, wherein the continuous non-linear channel comprises a continuous groove having an S-shape recessed in the outer surface.

Clause 32. The flow restriction device of any of clauses 29 to 31, wherein the fluid flowing from the distal end into the fluid collection device comprises blood and the fluid collection device comprises a blood collection device.

Clause 33. The flow restriction device of clause 32, wherein the blood collection device comprises a luer lock access device.

Clause 34. The flow restriction device of any of clauses 29 to 33, wherein the plug comprises an inner surface defining a lumen of the plug and the head portion comprises a normally-closed slit, and the normally-closed slit of the head portion is configured to prevent fluid flowing from the distal connector into the proximal connector via the lumen.

Clause 35. The flow restriction device of clause 34, wherein the plug comprises an inner surface defining a lumen of the plug and the head portion comprises a normally-closed slit, and when the normally-closed slit is subject to a distal fluid pressure, the normally-closed slit opens to allow fluid to flow from the proximal connector into the distal connector via the lumen.

Clause 36. The flow restriction device of clause 35, wherein the continuous non-linear channel comprises a first diameter, the lumen comprises a second diameter, and the first diameter is smaller than the second diameter.

Clause 37. The flow restriction device of clause 36, wherein the lumen defines an internal flowpath in which the fluid flows from the proximal connector into the catheter assembly via the distal connector.

Clause 38. The flow restriction device of clause 37, wherein the fluid flowing from the proximal connector into the catheter assembly comprises via the distal connector comprises an intravenous (IV) fluid.

Clause 39. A flow restriction device, comprising: a first connector comprising a female luer portion at a proximal end, a male luer portion at distal end, an internal surface defining an inner lumen of the first connector, and a compressible valve member mounted in the inner lumen, the first connector configured to couple to a fluid collection device; a second connector coupled to the male luer portion of the first connector and configured to couple to a catheter assembly, the second connector comprising an internal surface defining an inner lumen of the second connector; and a cannula mounted in the inner lumen of the second connector and extending therefrom into the female luer portion of the first connector, the compressible valve member mounted surrounding at least a portion of the cannula, wherein in a coupled configuration of the first connector and the fluid collection device, the compressible valve member is compressed by the fluid collection device to fluidly communicate the cannula and the fluid collection device via a slot of the compressible valve member.

Clause 40. The flow restriction device of clause 39, wherein the compressible valve member comprises a head portion including the slot, and a body portion extending distally from the head portion, and in the coupled configuration of the first connector and the fluid collection device, the body portion of the compressible valve member compresses to move the head portion distally to fluidly communicate a lumen of the cannula with the fluid collection device to allow a fluid to flow from the second connector into the fluid collection device via the lumen of the cannula.

Clause 41. The flow restriction device of clause 40, wherein the body portion comprises an accordion shape.

Clause 42. The flow restriction device of clause 40, wherein the head portion comprises a split-septum.

Clause 43. The flow restriction device of any of clauses 39 to 42, wherein the second connector further comprises a support portion extending radially inward from internal surface into the inner lumen of the second connector and comprising a mounting aperture, the cannula being mounted in the mounting aperture for fluidly communicating the second connector with the fluid collection device.

Clause 44. The flow restriction device of clause 43, wherein the cannula is press-fit in the support portion.

Clause 45. A flow restriction device, comprising: a first connector comprising a female luer portion having a lumen and disposed at a proximal end, a male luer portion having a lumen and disposed at distal end, and a compressible valve member mounted in the lumen of the female luer, the first connector configured to couple to a fluid collection device; a second connector comprising a female luer portion coupled to the male luer portion of the first connector and a male luer portion configured to couple to a catheter assembly, the female luer portion of the second connector comprising an inner lumen, and the male luer portion of the second connector comprising an inner lumen fluidly communicated with the inner lumen of the female luer portion of the second connector; and a post mounted in the second connector and extending from the inner lumen of the male luer portion of the second connector into an internal chamber of the compressible valve member via the lumen of the male luer portion of the first connector, the compressible valve member mounted surrounding at least a portion of the post, wherein in a coupled configuration of the first connector and the fluid collection device, the compressible valve member is compressed by the fluid collection device to fluidly communicate the post and the fluid collection device via a slot of the compressible valve member.

Clause 46. The flow restriction device of clause 45, wherein the post comprises an elongate tube having a lumen that tapers from a distal end to a proximal end of the post.

Clause 47. The flow restriction device of any of clauses 45 and 46, wherein the compressible valve member comprises a head portion including the slot, and a body portion extending distally from the head portion, and in the coupled configuration of the first connector and the fluid collection device, the body portion of the compressible valve member compresses to move the head portion distally to fluidly communicate a lumen of the post with the fluid collection device to allow a fluid to flow from the second connector into the fluid collection device via the lumen of the post.

Clause 48. The flow restriction device of clause 47, wherein the body portion comprises an accordion shape.

Clause 49. The flow restriction device of clause 47, wherein the head portion comprises a split-septum.

Clause 50. The flow restriction device of any of clauses 45 to 49, wherein the second connector further comprises a support portion at a proximal end of the male luer portion of the second connector, and the post is mounted on the support portion for fluidly communicating the second connector with the fluid collection device.

Clause 51. The flow restriction device of clause 50, wherein the support portion extends distally away from the male luer portion of the second connector into the lumen of the female luer portion of the second connector.

Clause 52. The flow restriction device of any of clauses 46 to 51, wherein the fluid flowing from the second connector into the fluid collection device via the lumen of the port comprises blood and the fluid collection device comprises a blood collection device.

Clause 53. A flow restriction device, comprising: a male luer connector portion configured to couple to a catheter assembly, the male luer connector portion comprising an internal surface defining a lumen thereof; a female luer connector portion disposed proximally to the male luer connector portion and configured to couple to a fluid collection device, the female luer connector portion comprising an internal surface defining a lumen fluidly connected to the lumen of the male luer connector portion; a tubing extending from the lumen of the male luer connector portion into the lumen of the female luer connector portion, wherein a lumen of the tube defines a flowpath along which a fluid flows from the male luer connector portion into the fluid collection device via the female luer connector portion.

Clause 54. The flow restriction device of clause 53, wherein at least a portion of the tubing comprises a non-linear tubing.

Clause 55. The flow restriction device of clause 54, wherein the portion of the non-linear tubing comprising a non-linear tubing is disposed in the lumen of the female luer connector portion.

Clause 56. The flow restriction device of any of clauses 54 and 55, wherein the non-linear tubing comprises a coil-shaped tubing.

Clause 57. The flow restriction device of any of clauses 54 to 55, wherein the non-linear tubing comprises an S-shaped tubing.

Clause 58. The flow restriction device of any of clauses 53 to 57, wherein the fluid flowing from the male luer connector portion into the fluid collection device via the female luer connector portion comprises blood and the fluid collection device comprises a blood collection device.

Clause 59. The flow restriction device of clause 58, wherein the blood collection device comprises a luer lock access device.

Clause 60. The flow restriction device of any of clauses 53 to 59, wherein the tubing comprises a linear tubing.

Clause 61. The flow restriction device of clause 60, wherein the linear tubing comprises a cannula mounted in the male luer connector portion.

Clause 62. The flow restriction device of clause 61, wherein the male luer connector portion further comprises a support portion extending radially inward from the internal surface into the lumen of the male luer connector portion and comprising a mounting aperture, the cannula being mounted in the mounting aperture for fluidly communicating the male luer connector portion with the fluid collection device.

Clause 63. The flow restriction device of clause 62, wherein the cannula is press-fit in the support portion.

Clause 64. The flow restriction device of any of clauses 60 to 63, wherein the fluid flowing from the male luer connector portion into the fluid collection device via the female luer connector portion comprises blood and the fluid collection device comprises a blood collection device.

Clause 65. The flow restriction device of clause 64, wherein the blood collection device comprises a luer lock access device.

Clause 66. A flow restriction device, comprising: a distal connector configured to couple to a catheter assembly, the distal connector comprising a first connection portion at a proximal end thereof and a second connection portion at a distal end thereof, each of the first and second connection portions comprising an inner surface defining a lumen; a proximal connector coupled to the distal connector and configured to couple to a fluid collection device; an insert mounted in the lumen of the first connection portion and comprising an outer surface having a groove recessed therein and extending at least partially along a length of the outer surface, wherein the groove is fluidly coupled to the lumen of the second connection portion and the lumen of the first connection portion, wherein the inner surface of the first connection portion encases the outer surface of the insert such that the groove and the inner surface of the first connection portion define at least a portion of a fluid channel along which fluid flows from the distal connector into the fluid collection device.

Clause 67. The flow restriction device of clause 66, wherein the groove comprises a linear groove recessed in the outer surface.

Clause 68. The flow restriction device of clause 67, wherein the outer surface further comprises longitudinally extending ledges, each disposed at opposing edges of the linear groove for sealing the edges.

Clause 69. The flow restriction device of clause 67, wherein the insert comprises a first channel section fluidly coupled to a proximal end of the lumen of the second connection portion, and a second channel section extending from the first channel section to the linear groove, the first and second channel sections and the linear groove together defining the fluid channel along which the fluid flows from the distal connector into the fluid collection device.

Clause 70. The flow restriction device of clause 69, wherein the first channel section and the linear groove are offset from each other in position, and the second channel section comprises a ramped surface coupling the first channel section to the linear groove.

Clause 71. The flow restriction device of any of clauses 66 to 70, wherein the fluid flowing from the distal end into the fluid collection device comprises blood and the fluid collection device comprises a blood collection device.

Clause 72. The flow restriction device of clause 71, wherein the blood collection device comprises a luer lock access device.

Clause 73. A flow restriction device, comprising: a connector comprising a body portion having a lumen and disposed at a proximal end, a base portion having a lumen and disposed at distal end, and a compressible valve member mounted on the base portion and extending into the lumen of the body portion, the body portion configured to couple to a fluid collection device; and a post having a lumen extending therethrough, the post mounted in the lumen of the base portion and extending into an internal chamber of the compressible valve member, the compressible valve member mounted surrounding the post, and the post configured to be fluidly communicated with a catheter assembly, wherein in a coupled configuration of the body portion and the fluid collection device, the compressible valve member is compressed by the fluid collection device to fluidly communicate the post and the fluid collection device via a slot of the compressible valve member.

Clause 74. The flow restriction device of clause 73, wherein the compressible valve member comprises a head portion including the slot, and a body portion extending distally from the head portion, and in the coupled configuration of the body portion and the fluid collection device, the body portion of the compressible valve member compresses to allow the head portion to move distally to fluidly communicate a lumen of the post with the fluid collection device and allow a fluid to flow from the distal end of the post into the fluid collection device via the lumen of the post.

Clause 75. The flow restriction device of clause 74 wherein the body portion comprises an accordion shape.

Clause 76. The flow restriction device of any of clauses 74 and 75, wherein the head portion comprises a split-septum.

Clause 77. The flow restriction device of any of clauses 73 to 76, wherein the lumen of the post tapers from a distal end to a proximal end of the post.

Clause 78. The flow restriction device of clause 77, wherein the fluid flowing from the distal end of the post into the fluid collection device via the lumen of the post comprises blood and the fluid collection device comprises a blood collection device.

Clause 79. A flow restriction device, comprising: a connector comprising a female luer portion having a lumen and disposed at a proximal end, a male luer portion having a lumen and disposed at distal end, and a compressible valve member mounted in the lumen of the female luer portion and fluidly coupled to the lumen of the male luer portion, the female luer portion configured to couple to a fluid collection device, and the male luer portion configured to couple to a catheter assembly; and a post mounted in the female luer portion and extending into an internal chamber of the compressible valve member, the compressible valve member mounted surrounding the post and the post being fluidly communicated with the lumen of the male luer portion, wherein in a coupled configuration of the female luer portion and the fluid collection device, the compressible valve member is compressed by the fluid collection device to fluidly communicate the post and the fluid collection device via a slot of the compressible valve member.

Clause 80. The flow restriction device of clause 79, wherein the compressible valve member comprises a head portion including the slot, and a body portion extending distally from the head portion, and in the coupled configuration of the female luer portion and the fluid collection device, the body portion of the compressible valve member compresses to move the head portion distally to fluidly communicate a lumen of the post with the fluid collection device and allow a fluid to flow from the male luer portion into the fluid collection device via the lumen of the post.

Clause 81. The flow restriction device of clause 80, wherein the body portion comprises an accordion shape.

Clause 82. The flow restriction device of any of clauses 80 and 81, wherein the head portion comprises a split-septum.

Clause 83. The flow restriction device of any of clauses 79 to 82, wherein the female luer portion further comprises a support portion at a distal end thereof, and the post is mounted on the support portion for fluidly communicating the lumen of the male luer portion with the fluid collection device.

Clause 84. The flow restriction device of clause 83, wherein the fluid flowing from the male luer portion into the fluid collection device via the lumen of the post comprises blood and the fluid collection device comprises a blood collection device.

Clause 85. A flow restriction device, comprising: a housing having a proximal end, an inner surface defining an internal chamber of the housing and a male luer portion defining a distal end of the housing and having a lumen fluidly communicated with the internal chamber, the internal chamber of the housing fluidly coupled to a connector; a slider reciprocally mounted in the internal chamber, the slider comprising a proximal end, a distal end, a mounting aperture extending from the proximal to the distal end of the slider, and a plurality of flow apertures extending from the proximal end to the distal end of the slider and surrounding the mounting aperture; a tubing having a lumen extending therethrough, the tubing mounted in the mounting aperture and extending from the proximal end of the slider distally through the distal end of the slider; and a spring member mounted in the internal chamber surrounding at least a portion of the tubing, wherein the spring member exerts a force to bias the proximal end of the slider against the inner surface of the housing to occlude fluid flow into the connector via the plurality of flow apertures, and to allow fluid flow into the connector via the tubing.

Clause 86. The flow restriction device of clause 85, wherein when the slider is subject to a distally-directioned fluid pressure, the slider is configured to move distally away from the inner surface of the housing to allow fluid to flow from the connector into the lumen of the male luer portion via both the tubing and the plurality of flow apertures.

Clause 87. The flow restriction device of clause 86, wherein the plurality of flow apertures comprises at least four flow apertures.

Clause 88. The flow restriction device of any of clauses 86 and 87, wherein the plurality of flow apertures extend longitudinally about an outer periphery of the slider.

Clause 89. The flow restriction device of any of clauses 85 to 88, wherein each of the plurality of flow apertures and the inner surface of the internal chamber define a flowpath through which the fluid flows from the connector into the lumen of the male luer portion when the slider is subject to the distally-directioned fluid pressure.

Clause 90. The flow restriction device of any of clauses 86 to 89, wherein at least a portion of the tubing comprises a longitudinal body uniformly extending in the internal chamber of the housing.

Clause 91. The flow restriction device of clause 90, wherein at least one of a diameter of the tubing at the proximal end and a diameter of the tubing at the distal end is greater than a diameter of the portion of the tubing which comprises a longitudinal body uniformly extending in the internal chamber of the housing.

Clause 92. The flow restriction device of any of clauses 90 and 91, wherein the tubing is flared radially outward from the portion of the tubing which comprises a longitudinal body uniformly extending in the internal chamber of the housing to at least one of the proximal and distal ends of the tubing.

Clause 93. The flow restriction device of any of clauses 86 to 92, wherein the slider comprises a polyisopropene seal material.

Clause 94. A blood collection system, comprising: a blood collection device comprising a container having an outer surface, an inner surface defining an internal chamber, a needle extending proximally from the inner surface, and a connection portion extending distally from the outer surface; and a flow restriction device fluidly coupled to the connection portion of the blood collection device, the flow restriction device comprising a cannula having an inner surface defining an internal flowpath therethrough, wherein the internal flowpath is fluidly coupled to a lumen of the needle for delivering blood drawn from a patient to the needle.

Clause 95. The blood collection system of clause 94, wherein the blood collection device comprises a luer lock access device.

Clause 96. The blood collection system of clause 94, wherein the blood collection device comprises a Vacutainer.

Clause 97. A blood collection system, comprising: a blood collection device comprising a container having an outer surface, an inner surface defining an internal chamber, and a connection portion extending distally from the outer surface and comprising a lumen extending therethrough; and a flow restriction device extending proximally from the inner surface and fluidly coupled to the lumen of the connection portion of the blood collection device, the flow restriction device comprising a cannula having an inner surface defining an internal flowpath therethrough, wherein the internal flowpath is fluidly coupled to the lumen of the connection portion for receiving blood drawn from a patient.

Clause 98. The blood collection system of clause 97, wherein the blood collection device comprises a luer lock access device.

Clause 99. The blood collection system of clause 97, wherein the blood collection device comprises a Vacutainer.

Clause 100. A blood collection system, comprising: a blood collection device comprising a container having an outer surface, an inner surface defining an internal chamber, a needle extending proximally from the inner surface, and a connection portion extending distally from the outer surface; and a flow restriction device engaged with the connection portion and fluidly coupled to a lumen of the needle, the flow restriction device comprising: a connector comprising a proximal end disposed in the connection portion, a distal end configured to couple to a catheter assembly, and an internal surface defining an inner lumen; and a cannula mounted in the inner lumen extending from the distal end of the connector into the connection portion, wherein a lumen of the cannula defines a flowpath along which blood flows from the distal end into the lumen of the needle.

Clause 101. The blood collection system of clause 100, wherein the connector further comprises a support portion disposed between the proximal and distal ends of the connector, the support portion comprising a proximal end, a distal end, and a central mounting aperture extending from the proximal to the distal end, the cannula mounted in the central mounting aperture for fluidly communicating the flow restriction device with the catheter assembly.

Clause 102. The blood collection system of any of clauses 100 and 101, wherein the blood collection device comprises a luer lock access device.

Clause 103. The blood collection system of any of clauses 100 to 102, wherein the blood collection device comprises a Vacutainer.

Clause 104. A blood collection system, comprising: a blood collection device comprising a container having an outer surface, an inner surface defining an internal chamber, a needle extending proximally from the inner surface, and a connection portion extending distally from the outer surface; and a flow restriction device engaged with the connection portion and fluidly coupled to a lumen of the needle, the flow restriction device comprising: a connector comprising a proximal end disposed in the connection portion, a distal end configured to couple to a catheter assembly, and an internal surface defining an inner lumen; and a cannula mounted in the inner lumen extending from the distal end to the proximal end of the connector, wherein a lumen of the cannula defines a flowpath along which blood flows from the distal end into the lumen of the needle.

Clause 105. The blood collection system of clause 104, wherein the connector further comprises a support portion disposed between the proximal and distal ends of the connector, the support portion comprising a proximal end, a distal end, and a central mounting aperture extending from the proximal to the distal end, the cannula mounted in the central mounting aperture for fluidly communicating the flow restriction device with the catheter assembly.

Clause 106. The blood collection system of any of clauses 104 and 105, wherein the blood collection device comprises a luer lock access device.

Clause 107. The blood collection system of any of clauses 104 to 106, wherein the blood collection device comprises a Vacutainer.

Clause 108. A blood collection system, comprising: a blood collection device comprising a container having an outer surface, an inner surface defining an internal chamber, a needle extending proximally from the inner surface, and a connection portion extending distally from the outer surface, wherein the connection portion comprises an insert portion including an outer surface having a continuous non-linear channel recessed therein; and a connector configured to couple to a catheter assembly, the connector comprising a first connection portion at a proximal end thereof and a second connection portion at a distal end thereof, and the first connection portion comprising an inner surface defining a lumen into which the insert portion is coupled, wherein the inner surface of the first connection portion encases the outer surface of the insert portion such that the continuous non-linear channel and the inner surface of the first connection portion define a non-linear fluid pathway along which a fluid flows from the connector into the lumen of the needle.

Clause 109. The blood collection system of clause 108, wherein the continuous non-linear channel comprises a continuous groove having a coil shape recessed in the outer surface.

Clause 110. The blood collection system of any of clauses 108 and 109, wherein the continuous non-linear channel comprises a continuous groove having an S-shape recessed in the outer surface.

Clause 111. The blood collection system of any of clauses 108 to 110, wherein the blood collection device comprises a luer lock access device.

Clause 112. The blood collection system of any of clauses 108 to 110, wherein the blood collection device comprises a Vacutainer.

Clause 113. The blood collection system of any of clauses 108 to 112, wherein the connection portion comprises a male luer connection portion.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A flow restriction device, comprising:
a distal connector configured to couple to a catheter assembly, the distal connector comprising a first connection portion at a proximal end thereof and a second connection portion at a distal end thereof, the first connection portion comprising an inner surface defining a lumen;
a proximal connector coupled to the distal connector and configured to couple to a fluid collection device, the proximal connector comprising an insert portion for inserting into the lumen of the first connection portion, the insert portion including an outer surface having a continuous non-linear channel recessed therein,
wherein the inner surface of the first connection portion encases the outer surface of the insert portion such that the continuous non-linear channel and the inner surface of the first connection portion define a non-linear fluid pathway along which a fluid flows from the distal connector into the fluid collection device.

2. The flow restriction device of claim 1, wherein the continuous non-linear channel comprises a continuous groove having a coil shape recessed in the outer surface.

3. The flow restriction device of claim 1, wherein the continuous non-linear channel comprises a continuous groove having an S-shape recessed in the outer surface.

4. The flow restriction device of claim 1, wherein the insert portion further comprises an inner surface defining a lumen of the insert portion, the lumen forming an internal flowpath along which a fluid flows from the proximal connector into the catheter assembly via the distal connector.

5. The flow restriction device of claim 4, further comprising a check valve disposed in the lumen of the insert portion, the check valve configured to (i) prevent fluid flowing from the distal connector into the proximal connector via the lumen of the insert portion, and (ii) allow fluid to flow from the proximal connector into the distal connector via the lumen of the insert portion.

6. The flow restriction device of claim 4, wherein the internal flowpath in which the fluid flows from the proximal connector into the catheter assembly via the distal connector comprises a linear flowpath.

7. The flow restriction device of claim 4, wherein the continuous non-linear channel comprises a first diameter, the internal flowpath comprises a second diameter, and the first diameter is smaller than the second diameter.

8. A flow restriction device, comprising:
a male luer connector portion configured to couple to a catheter assembly, the male luer connector portion comprising an internal surface defining a lumen thereof;
a female luer connector portion disposed proximally to the male luer connector portion and configured to couple to a fluid collection device, the female luer connector portion comprising an internal surface defining a lumen fluidly connected to the lumen of the male luer connector portion;
a tubing extending from the lumen of the male luer connector portion into the lumen of the female luer connector portion, wherein a lumen of the tube defines a flowpath along which a fluid flows from the male luer connector portion into the fluid collection device via the female luer connector portion.

9. The flow restriction device of claim 8, wherein at least a portion of the tubing comprises a non-linear tubing.

10. The flow restriction device of claim 9, wherein the portion of the non-linear tubing comprising a non-linear tubing is disposed in the lumen of the female luer connector portion.

11. The flow restriction device of claim 9, wherein the non-linear tubing comprises a coil-shaped tubing.

12. The flow restriction device of claim 9, wherein the non-linear tubing comprises an S-shaped tubing.

13. The flow restriction device of claim 8, wherein the tubing comprises a linear tubing.

14. The flow restriction device of claim 13, wherein the linear tubing comprises a cannula mounted in the male luer connector portion.

15. The flow restriction device of claim 14, wherein the male luer connector portion further comprises a support portion extending radially inward from the internal surface into the lumen of the male luer connector portion and comprising a mounting aperture, the cannula being mounted in the mounting aperture for fluidly communicating the male luer connector portion with the fluid collection device.

16. The flow restriction device of claim 15, wherein the cannula is press-fit in the support portion.

17. A flow restriction device, comprising:
a housing having a proximal end, an inner surface defining an internal chamber of the housing and a male luer portion defining a distal end of the housing and having a lumen fluidly communicated with the internal chamber, the internal chamber of the housing fluidly coupled to a connector;
a slider reciprocally mounted in the internal chamber, the slider comprising a proximal end, a distal end, a mounting aperture extending from the proximal to the distal end of the slider, and a plurality of flow apertures extending from the proximal end to the distal end of the slider and surrounding the mounting aperture;
a tubing having a lumen extending therethrough, the tubing mounted in the mounting aperture and extending from the proximal end of the slider distally through the distal end of the slider; and
a spring member mounted in the internal chamber surrounding at least a portion of the tubing, wherein the spring member exerts a force to bias the proximal end of the slider against the inner surface of the housing to occlude fluid flow into the connector via the plurality of flow apertures, and to allow fluid flow into the connector via the tubing.

18. The flow restriction device of claim 17, wherein when the slider is subject to a distally-directioned fluid pressure, the slider is configured to move distally away from the inner surface of the housing to allow fluid to flow from the connector into the lumen of the male luer portion via both the tubing and the plurality of flow apertures.

19. The flow restriction device of claim 18, wherein the plurality of flow apertures extend longitudinally about an outer periphery of the slider.

20. The flow restriction device of claim 17, wherein each of the plurality of flow apertures and the inner surface of the internal chamber define a flowpath through which the fluid flows from the connector into the lumen of the male luer portion when the slider is subject to the distally-directioned fluid pressure.
